(19) United States
(12) Patent Application Publication
(10) Pub. No.: US 2025/0025067 A1
(43) Pub. Date: Jan. 23, 2025

(54) SYSTEMS AND METHODS FOR MOTOR ASSESSMENT

(71) Applicant: Regeneron Pharmaceuticals, Inc., Tarrytown, NY (US)

(72) Inventors: Shawn Luke MISHRA, Houston, TX (US); Bharatkumar KOYANI, Cary, NC (US); Matthew F. WIPPERMAN, Brooklyn, NY (US); Oren LEVY, New York, NY (US); Yu BAI, Scarsdale, NY (US)

(73) Assignee: Regeneron Pharmaceuticals, Inc., Tarrytown, NY (US)

(21) Appl. No.: 18/777,757

(22) Filed: Jul. 19, 2024

Related U.S. Application Data
(60) Provisional application No. 63/514,644, filed on Jul. 20, 2023.

(51) Int. Cl. A61B 5/11; A61B 5/00

(52) U.S. Cl. CPC A61B 5/1124; A61B 5/7203; A61B 5/7267; A61B 5/7275

(57) ABSTRACT

Systems and methods for motor assessment including receiving first sensed signals in response to a first motor assessment performed using a first test device, receiving second sensed signals in response to a second motor assessment different than the first motor assessment and performed using the first test device, performing noise reduction for the first sensed signals and the second sensed signals, performing position normalization for the first sensed signals and the second sensed signals, generating transformed signals based on the noise reduction and the position normalization for the first sensed signals and the second sensed signals, extracting features based on the transformed signals, providing the extracted features to a machine learning model trained to output a motor assessment based prediction based on the transformed signals; and/or receiving the motor assessment based prediction from the machine learning model.

### CROSS-REFERENCE TO RELATED APPLICATIONS

This application claims priority to U.S. Provisional Application No. 63/514,644, filed on Jul. 20, 2023, the entirety of which is incorporated by reference herein.

### TECHNICAL FIELD

Embodiments disclosed herein are directed to systems and methods for quantitative motor assessment of rapid and/or alternating movements. Aspects of a smart device application for motor assessment based on noise reduction, positioning, feature extraction, and/or predictions related to rapid and/or alternating movements are also disclosed.

### INTRODUCTION

Traditional analysis for predicting a condition (e.g., a medical condition) onset, outcome, and/or trend is often conducted using complex devices in clinical settings. Such traditional analysis often requires large devices, one or more medical professionals to assist with conducting a test, and/or requires an individual to visit a clinical site to perform the testing. Simplified devices may be used to substitute for such traditional analysis. However, multiple devices are often required to capture variations in testing, and are often each limited to a single type of test.

This introduction section is provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

Aspects of the embodiments disclosed herein are directed to a method for motor assessment, the method including: receiving first sensed signals in response to a first motor assessment performed using a first test device; receiving second sensed signals in response to a second motor assessment different than the first motor assessment and performed using the first test device; performing noise reduction for the first sensed signals and the second sensed signals; performing position normalization for the first sensed signals and the second sensed signals; generating transformed signals based on the noise reduction and the position normalization for the first sensed signals and the second sensed signals; extracting features based on the transformed signals; providing the extracted features to a machine learning model trained to output a motor assessment based prediction based on the transformed signals; and receiving the motor assessment based prediction from the machine learning model.

One of the first motor assessment or the second motor assessment includes receiving a finger tapping input at the first test device. One of the first motor assessment or the second motor assessment includes receiving a finger tapping input at the first test device and wherein the finger tapping input is received at a touch screen of the first test device. One of the first motor assessment or the second motor assessment includes receiving a rotation-based input at the first test device. One of the first motor assessment or the second motor assessment includes receiving a rotation-based input at the first test device and wherein the rotation-based input includes a pronation component and a supination component. One of the first sensed signals or the second sensed signals indicate one or more of an area covered, a size, a force, or an impulse. One of the first sensed signals or the second sensed signals indicate one or more of an angular acceleration, an angular velocity, or a change in magnetic field.

The first sensed signals or the second sensed signals are generated using one or more sensors selected from a force sensor, a touch sensor, an accelerometer, a gyroscope, or a magnetometer. The first sensed signals or the second sensed signals are generated using an accelerometer, a gyroscope, and a magnetometer. The first sensed signals or the second sensed signals are generated using an accelerometer, a gyroscope, and a magnetometer and wherein noise reduction is performed based on an accelerometer signal generated at the accelerometer, a gyroscope signal generated at the gyroscope, and a magnetometer signal generated at the magnetometer. The first motor assessment is performed at a first time and the second motor assessment at a second time different from the first time. The second motor assessment includes performing multiple motor tasks. The second motor assessment includes performing multiple motor tasks, the second motor assessment including: performing a first motor task using the first test device at a first time; and performing a second motor task using a second test device at approximately the first time. The extracted features identify a patient waveform based on the first motor assessment and the second motor assessment. The motor assessment based prediction may include a key biomarker, a medical condition, an inclusion criteria, an exclusion criteria, a disease progression attribute, a disease regression attribute, a disease onset, a disease outcome, a disease trend, or a treatment. A trigger action that may include generating an updated motor assessment, triggering a repeat motor assessment, outputting a treatment, implementing a treatment, or modifying a database may be generated. One of the noise reduction or the position normalization is performed using a second machine learning model. The features are extracted using a second machine learning model.

Other aspects are directed to a method for motor assessment, the method including: receiving first sensed signals in response to a first motor assessment performed using a first test device; receiving second sensed signals in response to a second motor assessment different than the first motor assessment and performed using the first test device; extracting features based on a combination of the first sensed signals and the second sensed signals; providing the extracted features to a machine learning model trained to output a motor assessment based prediction based on the extracted features; and receiving the motor assessment based prediction from the machine learning model.

One of the first motor assessment or the second motor assessment includes receiving a finger tapping input at the first test device. One of the first motor assessment or the second motor assessment includes receiving a finger tapping input at the first test device and wherein the finger tapping input is received at a touch screen of the first test device. One of the first motor assessment or the second motor assessment includes receiving a rotation-based input at the first test device. One of the first motor assessment or the second motor assessment includes receiving a rotation-based input at the first test device and wherein the rotation-based input includes a pronation component and a supination component. One of the first sensed signals or the second sensed signals indicate one or more of an area covered, a size, a force, or an impulse. One of the first sensed signals or the second sensed signals indicate one or more of an angular acceleration, an angular velocity, or a change in magnetic field. The motor assessment based prediction may include a key biomarker, a medical condition, an inclusion criteria, an exclusion criteria, a disease progression attribute, a disease regression attribute, a disease onset, a disease outcome, a disease trend, or a treatment. A trigger action that may include generating an updated motor assessment, triggering a repeat motor assessment, outputting a treatment, implementing a treatment, or modifying a database may be generated.

The first sensed signals or the second sensed signals are generated using one or more sensors selected from a force sensor, a touch sensor, an accelerometer, a gyroscope, or a magnetometer. The first sensed signals or the second sensed signals are generated using an accelerometer, a gyroscope, and a magnetometer.

Other aspects are directed to a system including: a data storage device storing processor-readable instructions; and a processor operatively connected to the data storage device and configured to execute the instructions to perform operations that include: receiving first sensed signals in response to a first motor assessment performed using a first test device; receiving second sensed signals in response to a second motor assessment different than the first motor assessment and performed using the first test device; extracting features based on a combination of the first sensed signals and the second sensed signals; providing the extracted features to a machine learning model trained to output a motor assessment based prediction based on the extracted features; and receiving the motor assessment based prediction from the machine learning model.

One of the first motor assessment or the second motor assessment includes receiving a finger tapping input at the first test device. One of the first motor assessment or the second motor assessment includes receiving a finger tapping input at the first test device and wherein the finger tapping input is received at a touch screen of the first test device. One of the first motor assessment or the second motor assessment includes receiving a rotation-based input at the first test device. One of the first motor assessment or the second motor assessment includes receiving a rotation-based input at the first test device and wherein the rotation-based input includes a pronation component and a supination component. One of the first sensed signals or the second sensed signals indicate one or more of an area covered, a size, a force, or an impulse. One of the first sensed signals or the second sensed signals indicate one or more of an angular acceleration, an angular velocity, or a change in magnetic field.

The first sensed signals or the second sensed signals are generated using one or more sensors selected from a force sensor, a touch sensor, an accelerometer, a gyroscope, or a magnetometer. The first sensed signals or the second sensed signals are generated using an accelerometer, a gyroscope, and a magnetometer.

Other aspects are directed to a system including: a test device including a processor; an analysis model; and a machine learning framework trained to output a motor assessment based prediction based on extracted features, wherein the processor is configured to: generate first sensed signals in response to a first motor assessment performed using the test device, and generate second sensed signals in response to a second motor assessment performed using the test device, wherein the analysis model is configured to: extract the extracted features based on a combination of the first sensed signals and the second sensed signals, and provide the extracted features to the machine learning framework, and wherein the machine learning framework is configured to: output the motor assessment based prediction.

One of the first motor assessment or the second motor assessment includes receiving a finger tapping input at the test device. One of the first motor assessment or the second motor assessment includes receiving a finger tapping input at the test device and wherein the finger tapping input is received at a touch screen of the test device. One of the first motor assessment or the second motor assessment includes receiving a rotation-based input at the test device. One of the first motor assessment or the second motor assessment includes receiving a rotation-based input at the test device and wherein the rotation-based input includes a pronation component and a supination component. One of the first sensed signals or the second sensed signals indicate one or more of an area covered, a size, a force, or an impulse. One of the first sensed signals or the second sensed signals indicate one or more of an angular acceleration, an angular velocity, or a change in magnetic field.

The first sensed signals or the second sensed signals are generated using one or more sensors selected from a force sensor, a touch sensor, an accelerometer, a gyroscope, or a magnetometer. The first sensed signals or the second sensed signals are generated using an accelerometer, a gyroscope, and a magnetometer. The motor assessment based prediction may include a key biomarker, a medical condition, an inclusion criteria, an exclusion criteria, a disease progression attribute, a disease regression attribute, a disease onset, a disease outcome, a disease trend, or a treatment. A trigger action that may include generating an updated motor assessment, triggering a repeat motor assessment, outputting a treatment, implementing a treatment, or modifying a database may be generated. The machine learning framework may include a first machine learning model configured to extract the extracted features and a second machine learning model configured to output the trigger action.

The above summary is not intended to describe each and every embodiment or implementation of the present disclosure

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect and/or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1A is a system environment for motor assessment of movements, in accordance with aspects of the present disclosure.

FIG. 1B is a flow chart for motor assessment of movements, in accordance with aspects of the present disclosure.

FIG. 1C is another flow chart for motor assessment of movements, in accordance with aspects of the present disclosure.

FIG. 2A is a flow diagram for traditional motor assessment of movements.

FIG. 2B is a flow diagram for motor assessment of movements, in accordance with aspects of the present disclosure.

FIG. 3A is a flow diagram for multi-modal motor assessment, in accordance with aspects of the present disclosure.

FIG. 3B is a table for finger tapping tasks and durations, in accordance with aspects of the present disclosure.

FIG. 3C is a table for rotation tasks and durations, in accordance with aspects of the present disclosure.

FIGS. 3D-3G are tables for device properties for motor assessment, in accordance with aspects of the present disclosure.

FIG. 3H is a flow diagram for a digital multi-modal motor assessment, in accordance with aspects of the present disclosure.

FIG. 3I is a flow diagram for evaluations using motor assessment, in accordance with aspects of the present disclosure.

FIG. 3J is a table for finger tapping scoring, in accordance with aspects of the present disclosure.

FIG. 3K is a table for rotation scoring, in accordance with aspects of the present disclosure.

FIG. 3L is an example instruction for finger tapping, in accordance with aspects of the present disclosure.

FIG. 3M is an example instruction for rotation, in accordance with aspects of the present disclosure.

FIG. 3N shows example scoring for motor assessment, in accordance with aspects of the present disclosure.

FIGS. 4A-4B show example results for motor assessment, in accordance with aspects of the present disclosure.

FIG. 4C shows diagrams for a finger tapping assessments, in accordance with aspects of the present disclosure.

FIG. 4D is a table for tapping accuracy, in accordance with aspects of the present disclosure.

FIGS. 4E-4G show charts for noise reduction for motor assessment, in accordance with aspects of the present disclosure.

FIG. 4H shows charts for a rotation assessment, in accordance with aspects of the present disclosure.

FIGS. 5A-5D show results from rotation-based motor assessment, in accordance with aspects of the present disclosure.

FIG. 5E shows an example rotation motor assessment in real time, in accordance with aspects of the present disclosure.

FIG. 5F is a flow diagram for a finger tapping motor assessment, in accordance with aspects of the present disclosure.

FIG. 5G is a diagram for a finger tapping motor assessment, in accordance with aspects of the present disclosure.

FIG. 5H is a table for finger tapping tasks and durations, in accordance with aspects of the present disclosure.

FIG. 5I shows a rotation test device and associated movements, in accordance with aspects of the present disclosure.

FIG. 5J shows an example decomposition tree, in accordance with aspects of the present disclosure.

FIG. 5K shows a graphical representation of raw data for a rotation-based motor assessment, in accordance with aspects of the present disclosure.

FIG. 5L shows an example principal component analysis (PCA) plot, in accordance with aspects of the present disclosure.

FIG. 5M shows an example PCA plot for intermingled right and left hand data, in accordance with aspects of the present disclosure.

FIG. 5N shows an example projection plot corresponding to FIG. 5M, in accordance with aspects of the present disclosure.

FIG. 5O shows an example feature table, in accordance with aspects of the present disclosure.

FIG. 6 shows a z-score chart for motor assessment, in accordance with aspects of the present disclosure.

FIG. 7 is table for motor assessment benefits, in accordance with aspects of the present disclosure.

FIG. 8 is a flow diagram for training a machine learning model, in accordance with aspects of the present disclosure.

FIG. 9 is an example computing environment, in accordance with aspects of the present disclosure.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% in a stated numeric value.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Aspects of the disclosed subject matter are directed to receiving signals (e.g., user movement based signals) generated based on motion associated with a body component of an individual. The signals may be generated based on physical activity, electrical activity, positioning information, biometric data, movement data, or any attribute of an individual's body, an action associated with the individual's body, reaction of the individual's body, or the like. The signals may be generated by one or more test devices (e.g., mobile phone(s)) that may capture the signals using one or more sensors). For example, aspects of the disclosed subject matter are directed to methods for conducting quantitative motor assessment of movements, such as rapid movements and/or alternating movements (e.g., finger tapping, rotations, etc.) performed by a user. The user may perform such rapid and/or alternating (e.g., sequential) movements using one or more test devices and one or more sensors associated with the test devices may generate corresponding signals based on the movements. A software application activated using the one or more test devices may facilitate the motor assessments associated with the rapid and/or alternating movements.

According to implementations of the disclosed subject matter, a user may perform motor assessments using at least one test device. The motor assessments may include, for example, finger tapping, finger sliding, device rotation, device movement, and/or the like using one or more test devices. One or more sensors associated with the one or more test devices may detect parameters associated with the motor assessments. The parameters may be analyzed for noise reduction, positioning, feature extraction, and/or the like and may be transformed based on the analysis. The transformed parameters may be provided to a machine learning model that may categorize (e.g., based on clusters) the user and/or the user's movements and may be used to make predictions (e.g., disease onset, outcome, trend, etc.) for the user.

According to implementations of the disclosed subject matter, a user may perform multiple motor assessments using one or more test devices. The test devices may include or may otherwise be associated with one or more sensors that sense attributes associated with the motor assessments. For example, the user may use one or more mobile devices to perform the motor assessments. The mobile devices may include, for example, a force sensor, a touch sensors, heat sensors, visual sensors (e.g., cameras), radio frequency sensors, position sensors (e.g., an accelerometer, a gyroscope, a magnetometer, etc.), and/or any other applicable sensors configured to detect user actions based on the motor assessments.

According to an implementation, a motor assessment may be performed using an application (e.g., software application) activated using a mobile device. The application may be stored on and may be executed using the mobile device. The application may be stored and/or may be executed in communication with a remote component (e.g., a server, a database, a memory, etc.) which may be a cloud component. The application may provide a graphical user interface (GUI) that provides instructions and/or interfaces for implementing the motor assessments. The application may be in communication and/or receive sensed data from the one or more sensors. The application may facilitate performing the motor assessments via one or more interfaces provided via the GUI, as further discussed herein. The application may provide an interface directing a user to perform the motor assessments. According to an implementation, the user may perform the motor assessments using a test device separate from the mobile device used to provide the interface. Accordingly, the mobile device interface may provide instruction for a user to use a separate test device to perform the motor assessments. According to an implementation, both the mobile device and the separate test device may be used to perform the motor assessments.

The application may facilitate one or more motor assessments based on multiple sub-interfaces accessed via the application. The application may determine a user's dominant hand based on performance of one or more motor assessments or may receive dominant hand information from a user. The application may guide users through respective motor assessments using one or more interfaces, may provide visual and/or audio instructions, and may iteratively progress to different tasks and/or assessments automatically (e.g., in response to the completion of a task, in response to expiration of a duration of time, etc.). The application may provide reminders for incomplete tasks and may allow user-based customization (e.g., color customization, language customization, etc.).

According to an implementation, multiple motor assessments may be performed simultaneously or in sequence. For example, a first motor assessment may be performed at a first time and a second motor assessment may be performed at a subsequent second time. The first motor assessment and the second motor assessment may include performing the same task and/or may include performing different tasks (e.g., multiple finger motion tasks, multiple rotation-based tasks, etc.).

According to an example, a motor assessment may be a finger motion motor assessment. The finger motion motor assessment may include, for example, a finger tapping motion, a finger sliding motion, multiple finger motions, or any other motion performed by a user's one or more fingers for a given duration of time (e.g., approximately 5-20 seconds). A finger motion motor assessment may result in receiving rapid and/or alternating force or touch signals (e.g., based on a force sensor or a touch sensor) based on tapping received at a test device (e.g., a mobile device or other test device). A user may be provided an interface that visually indicates one or more target areas of the interface to rapidly and/or alternatively touch (e.g., alternating between two or more fingers, rapidly touching using one finger, etc.). The one or more target areas may remain the same throughout a motor assessment or may change during the duration of the motor assessment. The motor assessment may result in receiving the force or touch signals generated based on the touches (e.g., taps, slides, etc.) performed by the user. The force or touch signals may be received based on interaction with a component of the test device, such as a touch screen or other force detection interface (e.g., an interface connected to a force or touch sensor such as the back of a mobile phone). The touch screen may include a display to provide the interface to facilitate the finger motion motor assessment.

The finger motion motor assessment may include detecting properties associated with the force and/or touch associated with the finger motion. The properties may include, for example, amounts, durations, speeds, impulses, frequencies, forces, locations, accuracies, consistencies, and/or the like and/or derived properties based on or associated with the force and/or touch. Sensed signals generated by one or more sensors disclosed herein may be generated based on the respective properties of the force and/or touch.

According to an example, a motor assessment may be a rotation-based assessment. The rotation-based motor assessment may include, for example, one or more pronation rotations, one or more supination rotations, and/or any other rotation-based motion performed by a user for a given duration of time (e.g., approximately 5-20 seconds) performed using a test device (e.g., a mobile device or other test device). As used herein, pronation may refer to a rotational movement of a forearm that results in a user's palm facing posteriorly (e.g., when in an anatomic position) and may refer to movement performed with an arm being in an extended (e.g., fully extended) anatomical position. As used herein, supination may refer to a rotational movement of a forearm that results in a user's forearm facing anteriorly. The rotation-based motor assessment may result in receiving alternating rotation signals (e.g., based on an accelerometer, gyroscope, and/or magnetometer) based on a user rotating a test device. A user may be provided an interface that visually indicates one or more rotation-based motions for a user to perform (e.g., alternating between pronation and supination). The one or more rotation-based motions may remain the same throughout the motor assessment or may change during the duration of the motor assessment. The motor assessment may result in receiving the rotation signals generated based on the rotation-based motions performed by the user.

The rotation-based motion motor assessment may include detecting properties associated with the rotation associated with the rotation-based motion. The properties may include, for example, amounts, durations, frequencies, locations, accuracies, consistencies, angular acceleration, angular velocity, changes in magnetic fields and/or the like, and/or derivatives thereof associated with the rotation. Sensed signals generated by one or more sensors disclosed herein may be generated based on the respective properties of the rotations.

Although finger motion and rotation-based motion motor assessments are generally discussed herein, it will be understood that the techniques disclosed herein may apply to any motion based motor assessments associated with any user body part. For example, motor assessments may include hand motions, arm motions, finger motions, wrist motions, elbow motions, shoulder motions, neck motions, organ motion, body motion, waist motions, leg motions, knee motions, ankle motions, toe motions, and/or the like. The motions may be force-based, touch-based, rotation-based, yaw-based, lean-based, and/or the like.

According to implementations of the disclosed subject matter, sensed signals may be received based on the motor assessments. The sensed signals may be generated by the respective sensors configured to detect properties of the motions associated with the respective motion assessments. The sensed signals may be processed using a noise reduction module. The noise reduction module may be a software, hardware, and/or firmware module configured to modify the sensed signals. The noise reduction module may receive the sensed signals and transform the sensed signals based on one or more filters (e.g., high pass filters, low pass filters, band pass filters, etc.) and/or based on one or more other sensed signals.

The noise reduction module may perform sensor rectification to, for example, account for and/or mitigate sensor drift, sensor fusion, and/or the like. For example, multiple sensors may be used to detect a same motion property (e.g., angular rotation). The sensor signals from the multiple sensors may be compared to each other and a rectified signal for the given motion property may be generated based on the comparison. The rectified signal may transform the sensor signals to account for discrepancies, such as those caused by sensor drift. The rectified signal may be based on comparing differences between the respective sensed motion properties, as detected by the multiple sensors. The differences may be weighted based on each given sensor and the weighted differences may be compared to determine the rectified signal. The rectified signal may be generated based on, for example, comparing two or more signals from two or more respective sensors that indicate approximately a first motion property value being weighted higher than one or more other signals from a third sensor that indicates a second different motion property. According to this technique, a deviating sensor may be identified based on the comparison of the signals for the sensed motion property. According to an implementation, the deviating sensor may be recalibrated based on the comparison and subsequent signals from the deviating sensor may be generated based on the recalibration.

As an example, respective angular rotation motion signals may be generated by each of a gyroscope, an accelerometer, and a magnetometer associated with a test device. The respective motion signals may indicate an amount of rotation sensed by each of the gyroscope, accelerometer, and magnetometer. The respective motion signals may be compared to each other. If the result of the comparison is that the respective motion signals indicate approximately the same angular rotation motion (e.g., within a threshold deviation amount), then a rectified signal may be determined based on each of the respective motion signals (e.g., by averaging the respective motion signals). If the result of the comparison is that the respective motion signals indicate varying angular rotation motion, then the noise reduction module may perform a sensor rectification operation to generate a rectified signal. The rectification operation may be performed based on weighting the respective motion signals based on known good calibrations, overlapping motion signals (e.g., based on a threshold overlap between multiple respective motion signals), and/or the like.

According to an implementation, a rectification machine learning model may be trained to generate the rectification signal based on inputs including respective sensed signals from multiple sensors. The rectification machine learning model may be trained based on tagged or untagged training data that may include simulated or historical sensed data. The rectification machine learning model may be trained using supervised, unsupervised, and/or semi-supervised training. The rectification machine learning model may be training using machine learning algorithms disclosed herein. The rectification machine learning model may be trained to receive multiple motion signals and to compare the multiple motion signals to identify overlaps, outliers, and/or the like. The rectification machine learning model also have access to or otherwise receive historical motion signals from respective sensors such that it may determine a trend (e.g., a drift) over time. The rectification machine learning model may output the rectification signal based on the overlaps, outliers, trends, and/or the like.

The noise reduction module may further modify one or more sensed signals based on ambient properties associated with the one or more sensed signals. The ambient properties may include, but are not limited to, temperature, gravity, air properties, particulate properties, humidity, overall motion, material properties, and/or the like. An ambient property may have an effect on the sensed signals based on a user response based on the ambient property, a test device response based on the ambient property, and/or a sensor response based on the ambient property. For example, a temperature sensor may detect a test device and/or user temperature. The noise reduction module may normalize a sensed signal to account for deviations caused by the sensed temperature. As a specific example, an accelerometer may output sensed signals having a higher value when the sensed signal is detected while the respective test device experiences a temperature above a threshold temperature. The noise reduction module may generate a rectified signal to account for the higher value such that an adjusted (e.g., lower) value is indicated by the rectified signal. As another specific example, a motion sensor may detect an overall motion (e.g., when a motor assessment is conducted in a moving vehicle). The overall motion may be removed from sensed signals to account for the overall motion.

According to implementations of the disclosed subject matter, sensed signals may be received based on the motor assessments, as disclosed herein. The sensed signals may be generated by the respective sensors configured to detect properties of the motions associated with the respective motion assessments. The sensed signals may be processed using a position module. The position module may be a software, hardware, and/or firmware module configured to modify the sensed signals. The position module may receive the sensed signals and transform the sensed signals based on an absolute position calculated based on number of degrees of freedom (e.g., approximately 9 degrees of freedom, 13 degrees of freedom, etc.).

For example, multiple motion sensors (e.g., one or more of an accelerometer, a gyroscope, a magnetometer, etc.) may provide position signals for a motor assessment. The position signals may be used to determine a position based on, for example, nine degrees of freedom calculated using two or more of the position signals. As a specific example, an accelerometer, a gyroscope, and a magnetometer may each provide three degrees of freedom and each of the degrees of freedom may be used to calculate a zero position for the motor assessment. By applying the zero position any unintended deviation in position (e.g., user body movement during a rotation motion) may be removed from the position signals based on the motor assessment.

According to an implementation, quaternion coordinates may be generated based on position signals. The quaternion coordinates may describe orientation and/or rotation in three-dimensional space using an ordered set of four numbers. By using the quaternion coordinates, the position signals may be used to describe three-dimensional rotation about an arbitrary axis, without suffering from, for example, gimbal lock, unintended user motion, etc. The quaternion coordinates may be generated as a vector representation of the position and/or rotation of a test device during movement of the test device. The nine degrees of motion and the quaternion coordinates (e.g., thirteen degrees of motion) may be used to determine an absolute position of the test device such that any rotation or other movement is determined relative to the absolute position. Accordingly, the position signals may be transformed by the position module such that the resulting transformed rectification signals represent motion and position relative to the absolute position of a respective test device.

According to an implementation, a positioning machine learning model may be trained to generate the rectification signal based on inputs including respective position signals from multiple sensors. The positioning machine learning model may be trained based on tagged or untagged training data that may include simulated or historical sensed data. The positioning machine learning model may be trained using supervised, unsupervised, and/or semi-supervised training. The positioning machine learning model may be training using machine learning algorithms disclosed herein. The positioning machine learning model may be trained to receive multiple position signals and to compare the multiple position signals to identify multiple degrees of freedom and/or quaternion coordinates to determine an absolute position. The positioning machine learning model may output the rectification signal based on the multiple degrees of freedom and/or quaternion coordinates.

According to an implementation, a stationary position sensor (e.g., a visual sensor, a camera, a radio-frequency sensor, etc.) may provide a stationary position relative to an object. The object may be a stationary object such as, for example, the ground. The stationary position sensor may detect the object and use the location of the object relative to further determine an absolute position, as discussed herein.

According to an implementation, multiple test devises may be used simultaneously to perform a given motor assessment. For example, a rotation-based motor assessment may be performed with two test devices simultaneously. A user may perform a first task (a first rotation motion) with a first test device in her right hand and second task (a second rotation motion) with a second test device in her left hand. The first and second tasks may be performed at the same time. By using multiple test devices to perform multiple tasks simultaneously, sensed signals for respective tasks may be generated. The respective sensed signals may be compared to each other to extract comparative features, as further discussed herein. The multiple test devices may communicate with each other over a wired or wireless (e.g., Bluetooth) connection and may be synced based on the communication.

According to an implementation, a motor assessment performed in accordance with the techniques disclosed herein may provide a clinical outcome. The clinical outcome may include one or more of identifying biomarkers, screening patient populations, identification of inclusion criteria, identification of a disease progression attribute, identification of a disease regression attribute, identification of a treatment, predicting medical conditions/disease progression/treatment effects, identifying disease onset, identifying a disease outcome, or identifying a disease trend and/or the like.

For example, a group (e.g., cohort) of individuals may perform all or a subset of the motor assessments discussed herein. As a result of the motor assessments, condition states for each of the individuals may be determined. The condition states may include identification of a type of medical condition (e.g., a disease), a degree of medical condition, a progression of medical condition, an effect of a treatment, and/or the like. For any given condition state (e.g., identification of a type of medical condition), biomarkers for each individual may be identified (e.g., based on the extracted features). Of the identified biomarkers, biomarkers that meet an overlap threshold (e.g., correlation threshold) across the individuals may be identified as key biomarkers for the condition state. For example, a machine learning model, such as those discussed herein, may receive input data including the signals and/or extracted features discussed herein for each motor assessment for the individuals. The machine learning model may determine an overlap threshold based on the input data. The machine learning model may output the biomarkers (e.g., extracted features or biomarkers associated with the extracted features) that meet the overlap threshold. Accordingly, the key biomarkers that are most likely to indicate a given condition state (e.g., identification of a type of medical condition) may be identified.

Patient populations (e.g., for clinical trials, for treatment implementation, etc.) may be screened based on the identification of key biomarkers or associated extracted features. For example, a given individual may be identified as not having a key biomarker associated with a type of medical condition. As a result, this given individual may be excluded from a clinical trial associated with that medical condition, thereby efficiently reducing the sample size of the clinical trial.

A clinical outcome may include identification of disease progression. For example, an individual may perform the motor assessments discussed herein at two or more times. The extracted features associated with the temporally separated motor assessments may be compared to each other to determine the progression (or regression) of a disease. Such disease progression may be used to, for example, determine treatment(s), dosages, and/or changes to the same.

According to an implementation, a motor assessment performed in accordance with the techniques disclosed herein may trigger a subsequent action. The trigger action may include one or more of generating an updated motor assessment, triggering a repeat motor assessment, outputting a treatment, implementing a treatment, modifying a database, and/or the like.

As an example, the output of a motor assessment may be inconclusive due to user error, due to device calibration or error, and/or the like. As a result, the trigger action may include automatically activating a follow-up motor assessment via a device (e.g., a user device). As another example, the output of a motor assessment may require additional data to determine a clinical outcome. For certain medical conditions, an initial motor assessment may not provide sufficient data to determine a given clinical outcome (e.g., identifying biomarkers, screening patient populations, identification of inclusion criteria, identification of disease progression, identification of a treatment, or predicting medical conditions/disease progression/treatment effects, etc.). Accordingly, upon completion of the motor assessment(s), one or more updated motor assessments may be automatically activated via a device. As another example, an expected clinical outcome may be the determination of a fatigue onset for a given individual. Upon completion of one or more motor assessments by an individual, it may be determined that the point of fatigue onset is not identified. One or more updated motor assessments having a longer duration than the initial motor assessment(s) may be automatically activated via a device. The one or more updated motor assessments having the longer duration may enable determination of the point of fatigue onset as a clinical outcome. Accordingly, as exemplified herein, a trigger action may include automatically generating an updated medical assessment (e.g., having updated input requests, updated graphical interfaces, updated algorithms, updated logic, etc.) based on the result of an initial motor assessment.

As another example, a treatment may be output based on one or more motor assessments. The treatment may be automatically output my a machine learning model trained to receive, as inputs, signals or extracted features associated with the one or more motor assessments and output a treatment based on the input data. Such a machine learning model may be trained using training data that includes historical or simulated motor assessment signal or extracted feature information, historical or simulated treatments (e.g., including treatment type, dosage, duration, etc.), and/or historical or simulated treatment effects.

According to an implementation, a treatment determined based on the output of one or more motor assessments may be output by a system component. The system component may trigger the automatic administration of the treatment (e.g., a type of medication, a dosage of medication, etc.) via a medical device (e.g., by activating a software or hardware component of the medical device, via electronic communication with the medical device). Such automatic administration of the treatment may require detection of a consent flag (e.g., a software flag) at a processing component or memory component. Such a consent flag may be provided by a medical professional. For example, automatic administration of a treatment may be triggered and a request for consent may be electronically provided to a medical provider. The medical provider may provide the consent via an electronic device, such that the consent flag is triggered to allow automatic administration of the treatment (e.g., via a medical device). Absence of such a consent flag may prevent or delay such automatic administration of a treatment.

According to an implementation, transformed signals (e.g., rectification signals generated based on noise reduction and/or position normalization) may be used to extract features associated with the transformed signals. The extracted features may define a user waveform determined based on the user performing the motor assessments. The extracted features may be extracted by transforming raw data associated with the transformed signals into numerical features using a feature extraction machine learning model. The extracted features may correspond to properties associated with the motor assessments performed by the user. For example, the features may be extracted by correlating one or more of the transformed signal values with one or more other transformed signal values, with a time, with a user property, and/or the like.

A feature extraction machine learning model may be trained to extract the features based on inputs including the transformed signal values, one or more times, one or more user properties, and/or the like. The feature extraction machine learning model may be trained based on tagged or untagged training data that may include simulated or historical sensed data. The feature extraction machine learning model may be trained using supervised, unsupervised, and/or semi-supervised training. The feature extraction machine learning model may be training using machine learning algorithms disclosed herein. The feature extraction machine learning model may be trained to receive multiple transformed signals and to compare the multiple transformed signals to identify the features.

The extracted features may be based on given motor assessments and/or based on a combination of multiple motor assessments. For example, features may be extracted based on temporal characteristics such as motion (e.g., tapping) speed, variability, accuracy, consistency, duration, frequency, intervals (e.g., tap intervals), channel variation, asymmetry (e.g., rotation asymmetry), irregularities (e.g., in motion), entropy, multi-resolution analysis, and/or the like. Aggregate features may be generated based on multiple motor assessments (e.g., a finger motion assessment and a rotation-based assessment). The features may be based on any applicable relationship such as a mean values, median values, standard deviations, variance (e.g., interquartile range (IQR), minimum, maximum, etc.), linear modeling of cumulative motions, exploratory data analysis (EDA), and/or the like. Features may be based on a given user and/or may be based on global averages (e.g., based on a cohort of users).

The extracted features may be provided to a prediction machine learning model trained to make a prediction about the user based on the motor assessments. The prediction machine learning model may be trained to predict a medical condition diagnosis, a treatment for a condition, an improvement in a condition, a detrition of a condition, and/or the like. The prediction extraction machine learning model may be trained to make a prediction based on inputs including the extracted features. The prediction machine learning model may be trained based on tagged or untagged training data that may include simulated or historical sensed data. The prediction machine learning model may be trained using supervised, unsupervised, and/or semi-supervised training. The prediction machine learning model may be training using machine learning algorithms disclosed herein. The prediction machine learning model may be trained to receive multiple extracted features and to compare and/or correlate the multiple extracted features to make a prediction. The prediction machine learning model may make the prediction based on features associated with given motor assessments and/or based on a combination of motor assessments. The prediction machine learning model may output a cluster associated with the user based on the motor assessments. The cluster may categorize the user such that one or more predictions for the user are made based on the user's respective cluster. According to an implementation, users in a given cluster may be associated with the same or similar predictions.

The prediction may be output to the user and/or a third-party via a test device or a different device or component. For example, the prediction may be associated with a user account that is also associated with the software used to perform the motor assessments. The user may repeat motor assessments and/or perform new motor assessments over time and the prediction machine learning model may use prior motor assessment data to refine its predictions over time (e.g., based on one or more trends).

According to embodiments of the disclosed subject matter, extracted features that most contribute to a prediction output by a prediction machine learning (above a correlation threshold) may be identified as baseline covariates. According to an example, such baseline covariates may correspond to extracted features that most contributed (e.g., above a threshold that may be a numerical threshold or may be relative to other features) to modifying a weight, layer, bias, or synapse of a respective machine learning model during a training phase. As another example, such baseline covariates may correspond to extracted features that most contributed (e.g., above a threshold that may be a numerical threshold or may be relative to other features) to predicting a clinical outcome output by a production version of the prediction machine learning model. These baseline covariates may most contribute to a given outcome (e.g., motor function loss) associated with a clinical trial for a treatment effect for predicting the given outcome.

These baseline covariates may be used to screen potential clinical trial participants such that the corresponding clinical trial may be implemented in a more efficient manner. Screening potential clinical trial participants based on such identified baseline covariates may reduce the variability of the results of the clinical trial without biasing the same. Accordingly, screening for participants based on such baseline covariates may result in a more efficient clinical trial (e.g., by screening out participants based on such covariates), thereby reducing the sample size required for the clinical trial. Continuing the example discussed herein, identified baseline covariates may be used to screen (e.g., exclude) potential participants that are unlikely to experience motor function loss. The contemplated clinical trial outcome, according to this example, may be an effect on the degree of motor function loss based on a treatment effect (e.g., a drug, a medical device, therapy etc.). Accordingly, excluding participants that are unlikely to experience motor function loss may provide for a more relevant/efficient clinical trial.

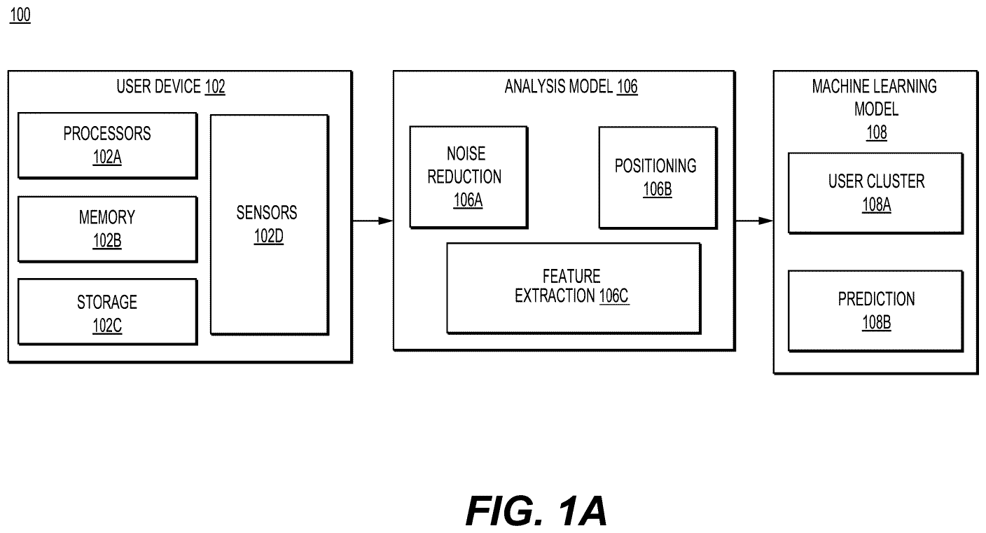

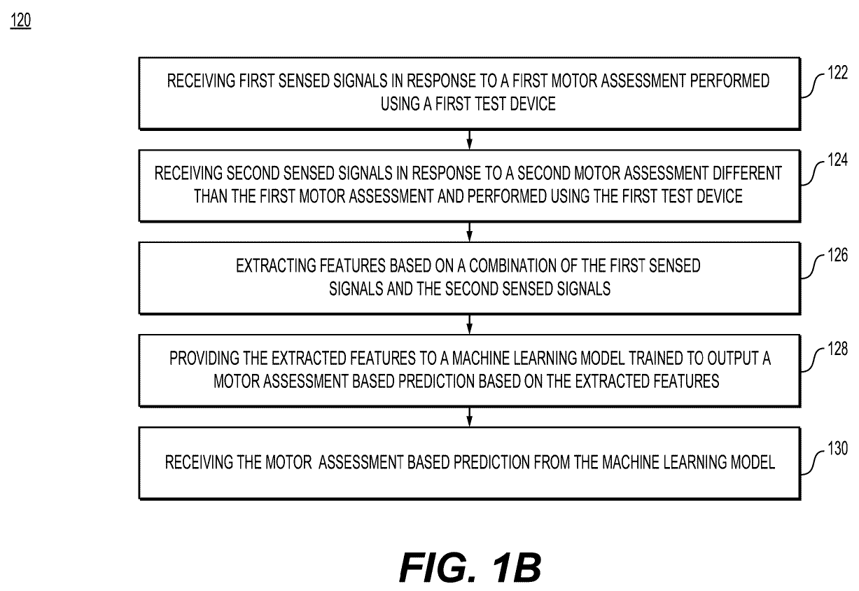

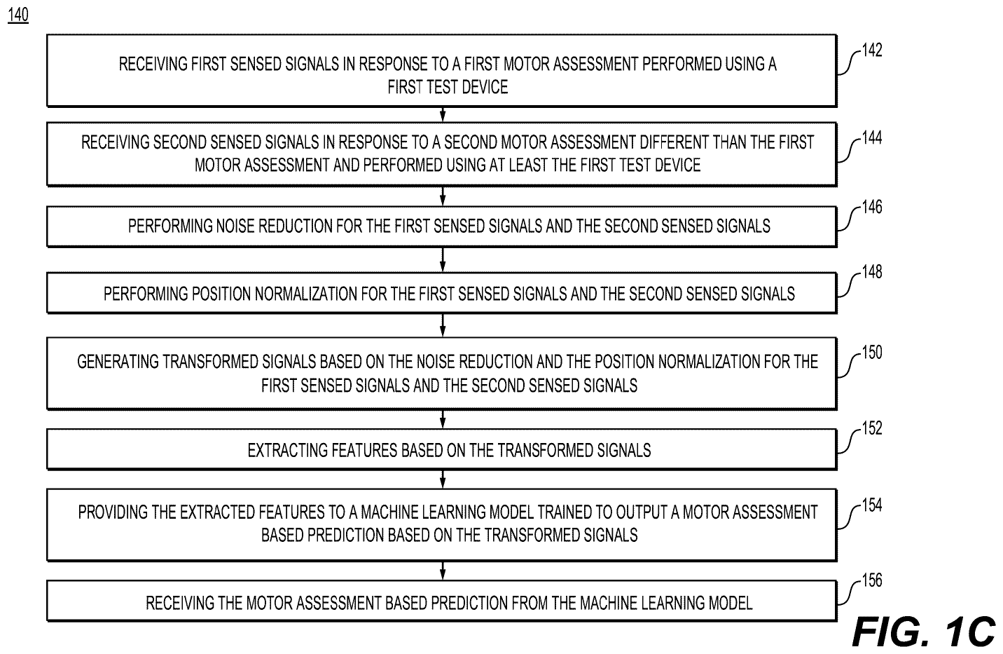

FIG. 1A shows a system environment 100 for motor assessments of movements in accordance with the subject matter disclosed herein. As shown, a user device 102 (e.g., a test device) may include one or more processors 102A, memories 102B, storage 102C, and/or sensors 102D. In some implementations, processors 102A may include one or more microprocessors, microchips, or application-specific integrated circuits. Memory 102B may include one or more types of random-access memory (RAM), read-only memory (ROM), and cache memory employed during execution of program instructions. Storage 102C may include one or more databases, cloud components, servers, or the like. Storage 102C may include a computer-readable, non-volatile hardware storage device that stores information and program instructions. Sensors 102D may be any sensors applicable to user device 102 and may include, but are not limited to, pressure sensors, motion sensors, cameras, biometric sensors, environment sensors, weight sensors, accelerometers, gyroscopes, magnetometers, and/or the like. Processors 102A may use data buses to communicate with memory 102B, storage 102C, and/or sensors 102D.

As shown in system environment 100, user device 102 may communicate with an analysis model 106. Analysis model 106 may be a standalone component, standalone software, or may be a part of user device 102, user device 102 software, and/or may be in communication with user device 102. For example, user device 102 may communicate with analysis model 106 over a network such that analysis model 106 is a cloud component or stored at a cloud component. Analysis model 106 may be implemented using one or more processors, memory, storage, or the like. According to an implementation, analysis model 106 may receive data generated using sensors 102D. Analysis model 106 may receive the data directly from user device 102 (e.g., over a network) or may receive the data through a different component that receives the data from user device 102 and/or another device, system, or component.

Analysis model 106 may include one or more components such as noise reduction module 106A, positioning module 106B, feature extraction module 106B, and/or the like. The one or more components may be implemented as software components, hardware components, and/or firmware components. Noise reduction module 106A may be used to implement the noise reduction techniques disclosed herein. Positioning module 106B may be used to implement the positioning techniques disclosed herein. Feature extraction module 106C may be used to implement the feature extraction techniques disclosed herein.

As shown in system environment 100, analysis module 106 may communicate with a machine learning model 108. Machine learning model 108 may be a standalone component, standalone software, or may be a part of user device 102, user device 102 software, and/or may be in communication with user device 102. For example, analysis module 106 may communicate with machine learning model 108 over a network such that machine learning model 108 is a cloud component or stored at a cloud component. Machine learning model 108 may be implemented using one or more processors, memory, storage, or the like. According to an implementation, machine learning model 108 may receive data output by analysis model 106. Machine learning model 108 may receive the data directly from analysis model 106 (e.g., over a network) or may receive the data through a different component that receives the data from analysis model 106, user device 102, and/or another device, system, or component.

Machine learning model 108 may include one or more components such as user cluster module 108A, prediction module 108B, and/or the like. The one or more components may be implemented as software components, hardware components, and/or firmware components.

FIG. 1B shows a flowchart 120 for motor assessment based predictions, in accordance with the subject matter disclosed herein. At step 122, first sensed signals may be received in response to a first motor assessment performed using a first test device (e.g., user device 102). At step 124, second sensed signals may be received in response to a second motor assessment performed using the first test device (e.g., user device 102). The first motor assessment and the second motor assessment may be performed sequentially using the same first test device. A user may activate a software application using user device 102. The software application may guide the user to perform the first motor assessment and the second motor assessment in accordance with the techniques disclosed herein. For example, a user may be provided visual interface (e.g., a GUI) that directs the user to perform the first and second motor assessments. The visual interface may direct the user to perform movement based actions (e.g., finger motion actions, rotation-based actions, etc.). The user may provide inputs (e.g., perform the movement based actions) in accordance with the visual interface direction. Sensors 102D may sense movement information in response to the movement based actions performed by the user, as disclosed herein.

The sensed data may be generated at the one or more sensors 102D that may be part of a device or a system. The sensed data may be provided by processors 102A and may be stored at memory 102B and/or storage 102C such that processors 102A may retrieve the sensed data from memory 102B and/or storage 102C. The sensed data may be in the format output by one or more sensors 102D or may be in a different format. For example, processors 102A may receive the sensed data in a first format and may convert the sensed data to a second format.

At step 126, features may be extracted based on a combination of the first sensed signals and the second sensed signals. The features may be extracted in accordance with the techniques disclosed herein. The extracted features may include features determined based on both the first motor assessment and the second motor assessment. Accordingly, the extracted features may be based on the first sensed signals received at step 122 and the second sensed signals received at step 124. By extracting features based on both the first motor assessment and based on the second motor assessment, attribute associated with overlaps, relationships, and/or correlations between the first motor assessment and the second motor assessment may be incorporated by the extracted features.

At step 128, the features extracted at step 126 may be provided to a machine earning mode, such as a prediction machine learning model discussed herein. The machine learning model may be trained to output a motor assessment based prediction based on the features extracted at step 126, as discussed herein. The machine learning model may apply the extracted features to one or more weights, layers, biases, synapses, nodes, and/or the like configured based on training the machine learning model.

At step 130, the machine learning model may output a motor assessment based prediction based on both the first motor assessment and the second motor assessment. The prediction may include one or more of a disease onset, outcome, or trend such as, for example, a medical condition diagnosis, a treatment for a condition, an improvement in a condition, a detrition of a condition, and/or the like, as discussed herein. As discussed herein, key biomarkers may be determined based on the extracted features by, for example, determining which extracted features contributed most to generating the machine learning model output. Additionally, as discussed herein, clinical outcomes and/or trigger actions may be determined based on the extracted features and/or the output of the machine learning model.

Accordingly, based on the techniques associated with system environment 100, a user may use user device 102 to perform multiple motor assessments. Sensors 102D associated with user device 102 may generate sensed signal based on the multiple motor assessments. The multiple motor assessments may be performed during a same software application session (e.g., simultaneously) using the same user device 102 such that any unintended effects from a variability in duration of time, from variability from distinct software application sessions, from variability in device properties, from variability in sensor properties, and/or the like may be eliminated or substantially mitigated.

FIG. 1C shows a flowchart 140 for motor assessment based predictions, in accordance with the subject matter disclosed herein. At step 142, first sensed signals may be received in response to a first motor assessment performed using a first test device (e.g., user device 102). At step 144, second sensed signals may be received in response to a second motor assessment performed using the first test device (e.g., user device 102). The first motor assessment and the second motor assessment may be performed sequentially using the same first test device. A user may activate a software application using user device 102. The software application may guide the user to perform the first motor assessment and the second motor assessment in accordance with the techniques disclosed herein. For example, a user may be provided visual interface (e.g., a GUI) that directs the user to perform the first and second motor assessments. The visual interface may direct the user to perform movement based actions (e.g., finger motion actions, rotation-based actions, etc.). The user may provide inputs (e.g., perform the movement based actions) in accordance with the visual interface direction. Sensors 102D may sense movement information in response to the movement based actions performed by the user, as disclosed herein.

The sensed data may be generated at the one or more sensors 102D that may be part of a device or a system. The sensed data may be provided by processors 102A and may be stored at memory 102B and/or storage 102C such that processors 102A may retrieve the sensed data from memory 102B and/or storage 102C. The sensed data may be in the format output by one or more sensors 102D or may be in a different format. For example, processors 102A may receive the sensed data in a first format and may convert the sensed data to a second format.

At step 146, noise reduction may be performed for the first sensed signals received at 142 and for the second sensed signals received at 144, in accordance with the techniques disclosed herein. The noise reduction may be performed by analysis model 106 via noise reduction module 106A. The noise reduction may be performed using one or more filters and/or based on one or more if the first sensed signals received at 142 and for the second sensed signals received at 144, in accordance with the techniques disclosed herein.

At step 148, positioning normalization may be performed for the first sensed signals received at 142 and for the second sensed signals received at 144, in accordance with the techniques disclosed herein. The positioning normalization may be performed by analysis model 106 via positioning module 106B. The positioning normalization may be performed using, for example, multiple degrees of freedom (e.g., approximately 9 degrees of freedom, approximately 13 degrees of freedom, etc.). The multiple degrees of freedom may be determined based on multiple position signals generated by multiple sensors, based on quaternion coordinates, and/or based a stationary position (e.g., determined based on one or more stationary position sensors).

At step 150, transformed signals may be generated based on the noise reduction at step 146 and the position normalization at step 148. The transformed signals may be modified versions of the first sensed signals received at step 142 and the second sensed signals received at step 144, in accordance with techniques disclosed herein.

At step 152, features may be extracted based on the transformed signals generated at step 150. The features may be based on a combination of the first sensed signals received at step 142 and the second sensed signals received at step 144. The features may be extracted in accordance with the techniques disclosed herein. The extracted features may include features determined based on both the first motor assessment and the second motor assessment. Accordingly, the extracted features may be based on the first sensed signals received at step 142 and the second sensed signals received at step 144. By extracting features based on both the first motor assessment and based on the second motor assessment, attribute associated with overlaps, relationships, and/or correlations between the first motor assessment and the second motor assessment may be incorporated by the extracted features.

At step 154, the features extracted at step 152 may be provided to a machine earning model. For example, the features extracted at step 152 may be provided to a prediction machine learning model as discussed herein. The machine learning model may be trained to output a motor assessment based prediction based on the features extracted at step 152, as discussed herein. The machine learning model may apply the extracted features to one or more weights, layers, biases, synapses, nodes, and/or the like configured based on training the machine learning model.

At step 156, the machine learning model may output a motor assessment based prediction based on the extracted features. As discussed, the extracted features may be based on both the first motor assessment and the second motor assessment. The prediction may include one or more of a medical condition diagnosis, a treatment for a condition, an improvement in a condition, a detrition of a condition, and/or the like, as discussed herein. As discussed herein, key biomarkers may be determined based on the extracted features by, for example, determining which extracted features contributed most to generating the machine learning model output. Additionally, as discussed herein, clinical outcomes and/or trigger actions may be determined based on the extracted features and/or the output of the machine learning model.

Accordingly, based on the techniques associated with flowchart 140, a user may use a user device 102 to perform multiple motor assessments. Sensors 102D associated with user device 102 may generate sensed signal based on the multiple motor assessments. The multiple motor assessments may be performed during a same software application session (e.g., simultaneously) using the same user device 102 such that any unintended effects from a variability in duration of time, from variability from distinct software application sessions, from variability in device properties, from variability in sensor properties, and/or the like may be eliminated or substantially mitigated.

Each block in the system diagram of FIG. 1A or flowcharts of FIG. 1B or 1C can represent a module, segment, or portion of program instructions, which includes one or more computer executable instructions for implementing the illustrated functions and operations. In some alternative implementations, the functions and/or operations illustrated in a particular block of a flow diagram or flowchart can occur out of the order shown in the respective figure.

For example, two blocks shown in succession can be executed substantially concurrently, or the blocks can sometimes be executed in the reverse order, depending upon the functionality involved. It will also be noted that each block of the flow diagram and combinations of blocks in the block can be implemented by special purpose hardware-based systems that perform the specified functions or acts, or combinations of special purpose hardware and computer instructions.

In various implementations disclosed herein, systems and methods are described for using machine learning to, for example, perform noise reduction, perform position normalization, extract features, and/or make predictions. By training a machine learning model, e.g., via supervised or semi-supervised learning, to learn associations between training data and ground truth data, the trained machine learning model may be used to validate one or more test devices.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, extreme gradient boosting (XGBoost), random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

As discussed herein, machine learning techniques may include one or more aspects according to this disclosure, e.g., a particular selection of training data, a particular training process for the machine learning model, operation of a particular device suitable for use with the trained machine learning model, operation of the machine learning model in conjunction with particular data, modification of such particular data by the machine learning model, etc., and/or other aspects that may be apparent to one of ordinary skill in the art based on this disclosure.

Generally, a machine learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine learning model, e.g., compare the output of the trained model with the ground truth for that portion of the training data to evaluate an accuracy of the trained model. The training of the machine learning model may be configured to cause the machine learning model to learn associations between training data and ground truth data, such that the trained machine learning model is configured to determine an output in response to the input data based on the learned associations.

In various implementations, the variables of a machine learning model may be interrelated in any suitable arrangement in order to generate the output. For example, in some embodiments, the machine learning model may include image-processing architecture that is configured to identify, isolate, and/or extract features, geometry, and or structure in one or more of the medical imaging data and/or the non-optical in vivo image data. For example, the machine learning model may include one or more convolutional neural network (“CNN”) configured to identify features in data, and may include further architecture, e.g., a connected layer, neural network, etc., configured to determine a relationship between the identified features in order to determine a location in the data.

In some instances, different samples of training data and/or input data may not be independent. Thus, in some embodiments, the machine learning model may be configured to account for and/or determine relationships between multiple samples.

For example, in some embodiments, the machine learning models described herein may include a Recurrent Neural Network (“RNN”). Generally, RNNs are a class of feed-forward neural networks that may be well adapted to processing a sequence of inputs. In some embodiments, the machine learning model may include a Long Shor Term Memory (“LSTM”) model and/or Sequence to Sequence (“Seq2Seq”) model. An LSTM model may be configured to generate an output from a sample that takes at least some previous samples and/or outputs into account. A Seq2Seq model may be configured to, for example, receive a sequence of non-optical in vivo images as input, and generate a sequence of locations, e.g., a path, in the medical imaging data as output.

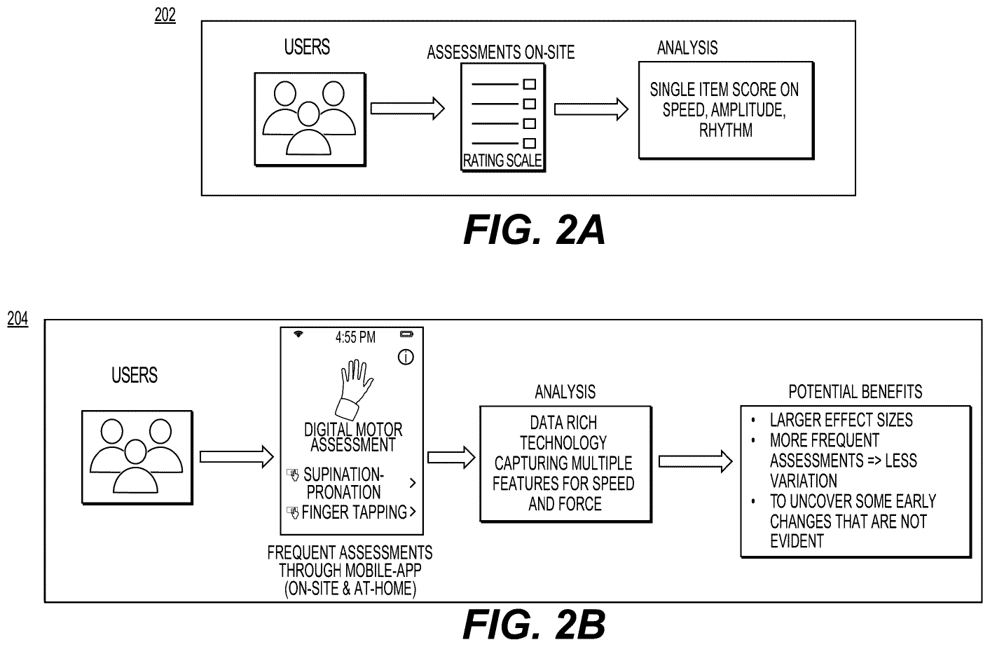

FIG. 2A is a flow diagram 202 for traditional motor assessment of movements. As shown in flow diagram 202, traditionally, users may visit an assessment location for an on-site assessment of motor functions. The assessments may be conducted using multiple devices, where the output of each device is analyzed individually to determine an item score based on speed, amplitude and rhythm associated with movement.

FIG. 2B is a flow diagram 204 for motor assessment of movements in accordance with the techniques disclosed herein. As shown in flow diagram 204, users may perform frequent motor assessments using a mobile application. The users may perform the motor assessments at any location of their choosing by accessing a software application via user device 102 in accordance with the techniques disclosed herein. User device 102 may be used to perform the motor assessments such that multiple features, as discussed herein, are extracted based on the respective sensor data generated based on the motor assessments. Accordingly, the techniques disclosed herein provide benefits including large effect sizes, more frequent assessments resulting in less variation, and/or predictions and early assessments.

In accordance with techniques disclosed herein, assessments may be made daily, twice weekly, weekly, biweekly, and/or monthly as discussed in Rutkove et al. Improved ALS clinical trials through frequent at-home self-assessment: a proof of concept study. Annals of Clinical and Translational Neurology 7(7): 1148-1157 (2020), which is incorporated by reference herein. More frequent assessments, as made possible in accordance with the techniques disclosed herein, may reduce sample sizes required to detect signals and/or relevant features in clinical trials. Assessments may be categorized, for example, based on sample size, mean, standard deviation, and/or effect size data for example experiments related to slow vital capacity (SVC), activity tracker(s), and/or amyotrophic lateral sclerosis (ALS) functional rating scale (ALSFRS-R).

Average Unified Parkinson's Disease Rating Scale (UPDRS) scores (e.g., for bradykinesia severity) may be determined using a finger motion (e.g., finger tapping) motor assessment, as disclosed in Lee et al. A Validation Study of a Smartphone-Based Finger Tapping Application for Quantitative Assessment of Bradykinesia in Parkinson's Disease. PLoS ONE 11(7): e0158852 (2016), which is incorporated by reference herein. For example, finger tapping speed may correlate with bradykinesia severity in users Parkinson's disease (PD). Corresponding scores may, for example, be used by a prediction machine learning model to determine PD based predictions based on a finger tapping based extracted feature, as discussed herein.

Finger tapping variability verses Movement Disorder Society-Sponsored Revision UPDRS (MDS-UPDRS) scores may be determined based on a finger tapping motor assessment, as disclosed in Lipsmeier et al. Reliability and validity of the Roche PD Mobile Application for remote monitoring of early Parkinson's disease. Sci. Rep. 12:12081 (2022), which is incorporated by reference herein. Proration and supination speed verses MDS-UPDRS scores may be based on a rotation-based motor assessment. Data associated with such scores (e.g., based on a finger tapping motor assessment and/or a rotation-based motor assessment) may, for example, be used by a prediction machine learning model to determine PD based predictions based on a finger tapping and/or rotation-based extracted feature, as discussed herein.

### Experimental Study

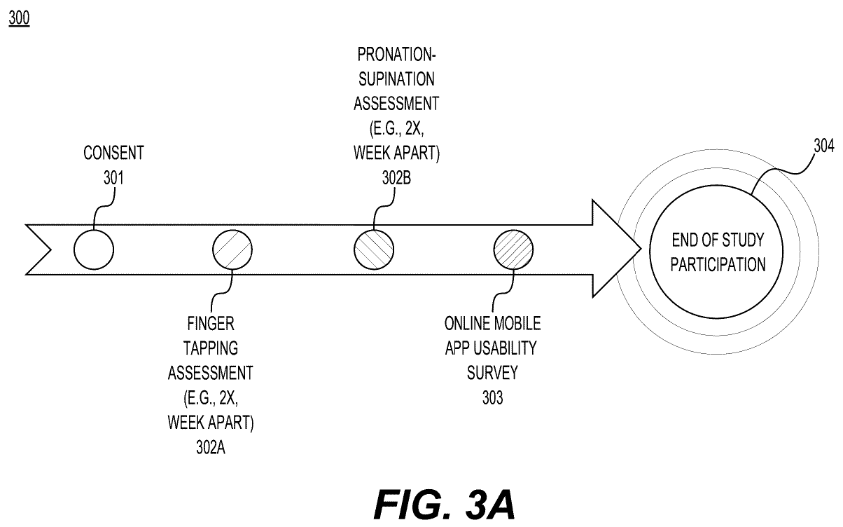

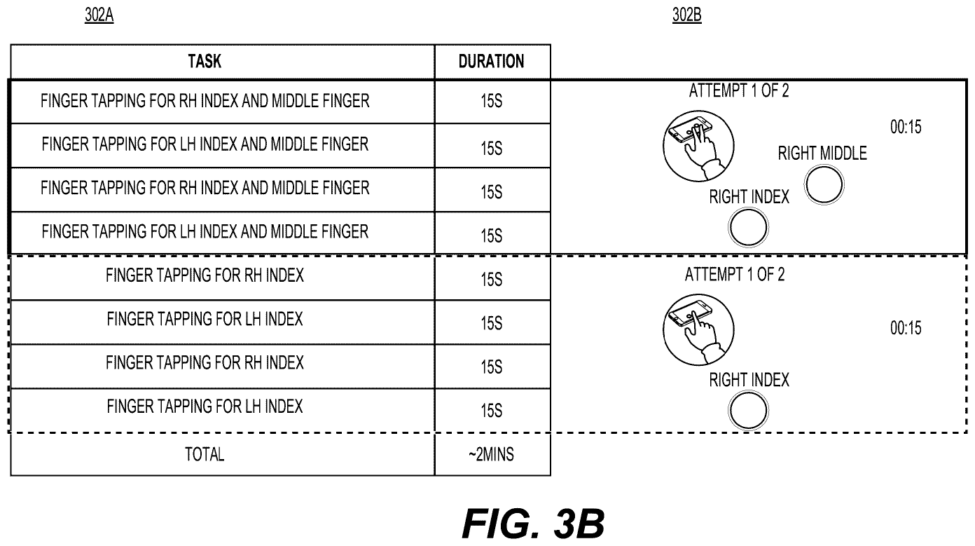

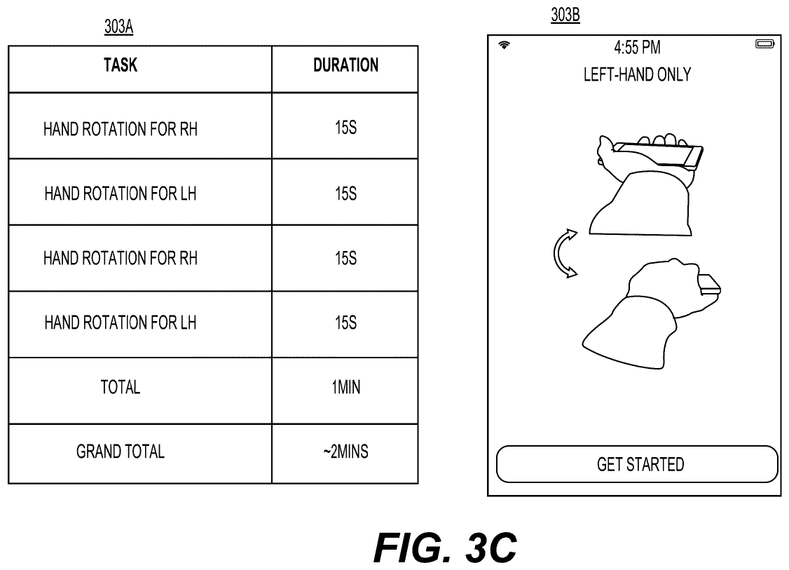

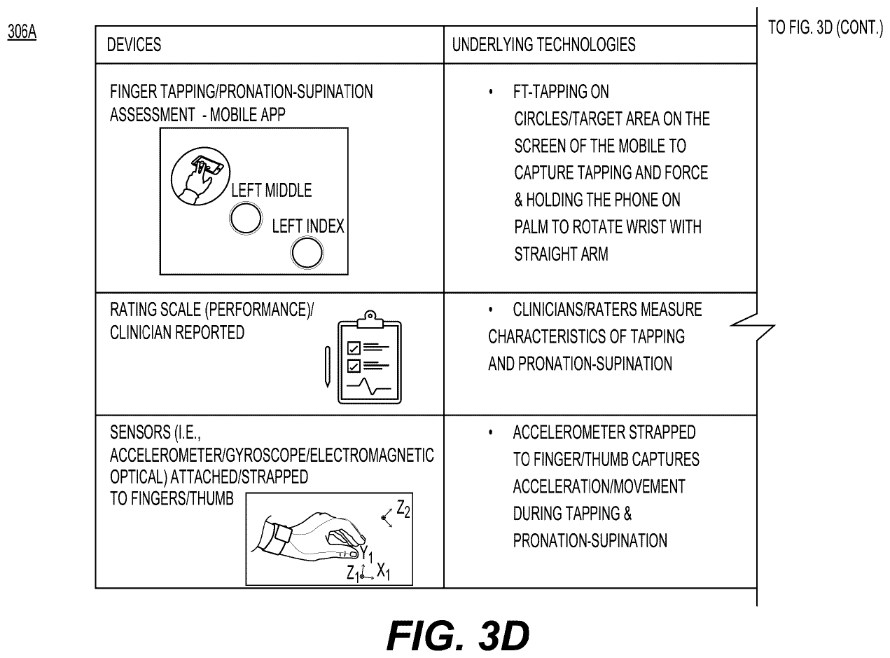

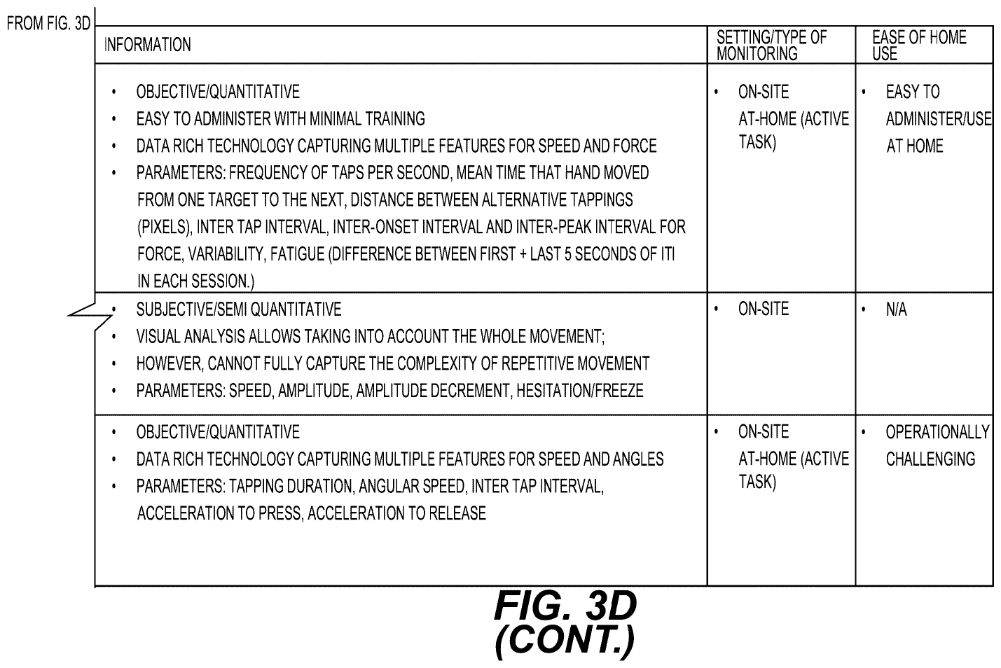

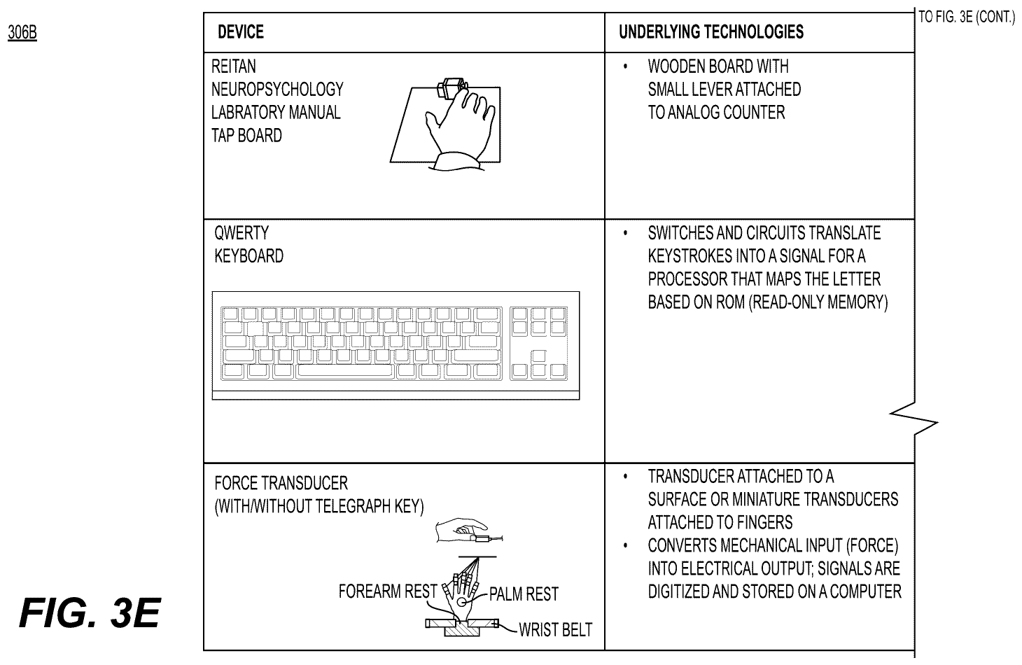

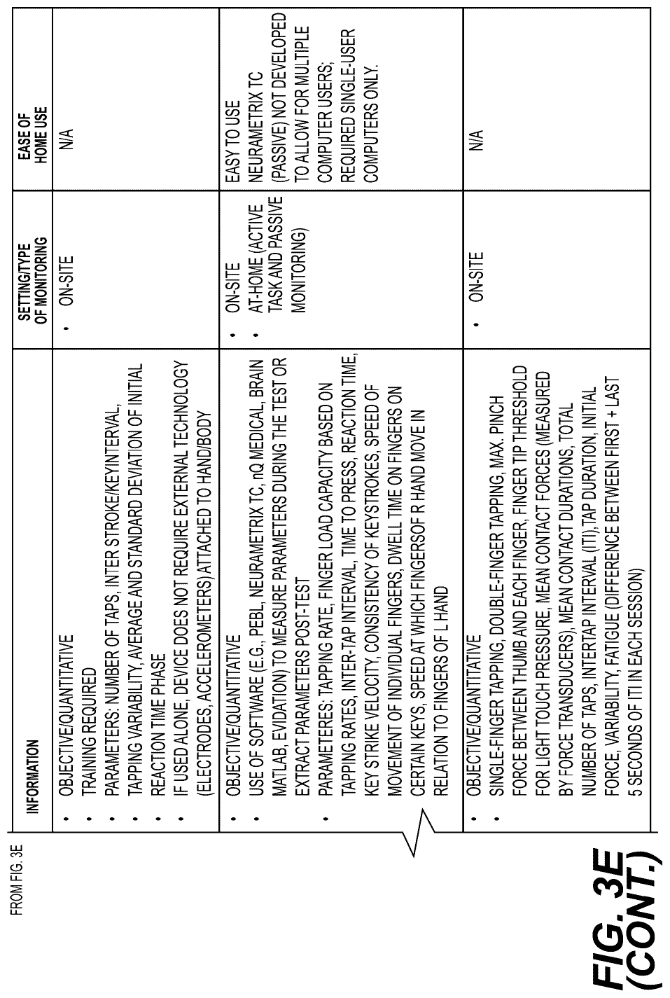

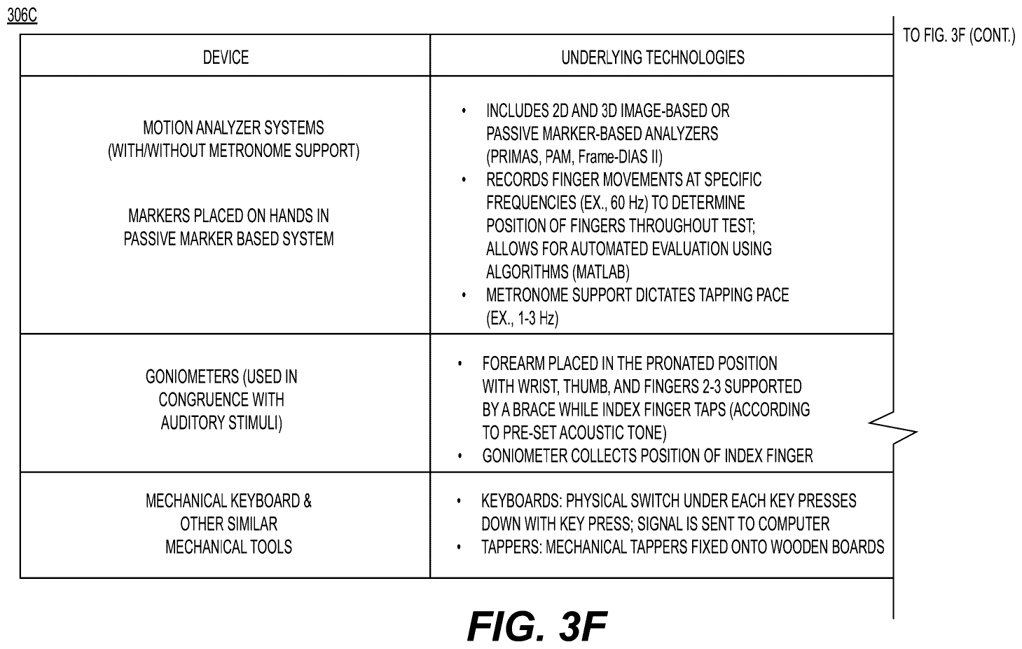

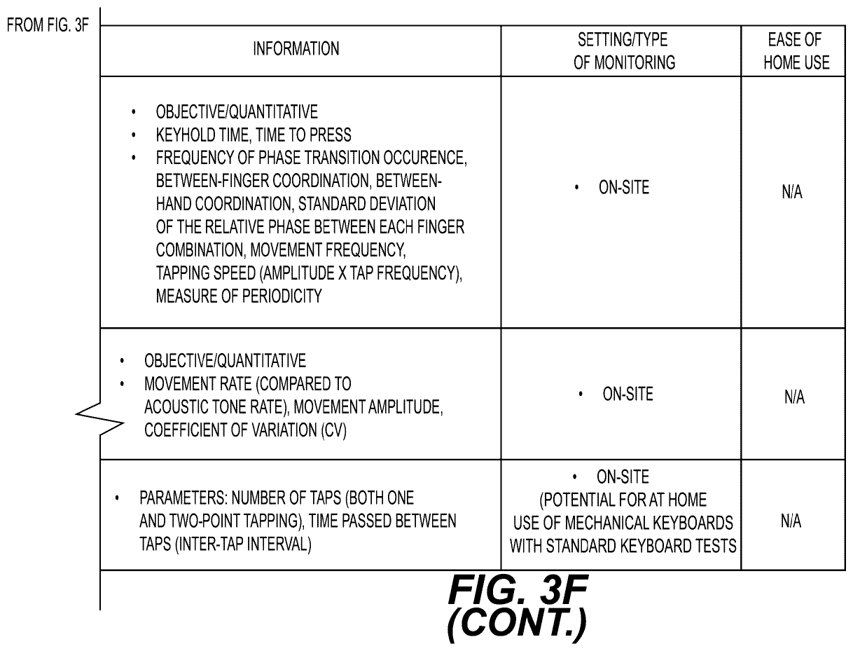

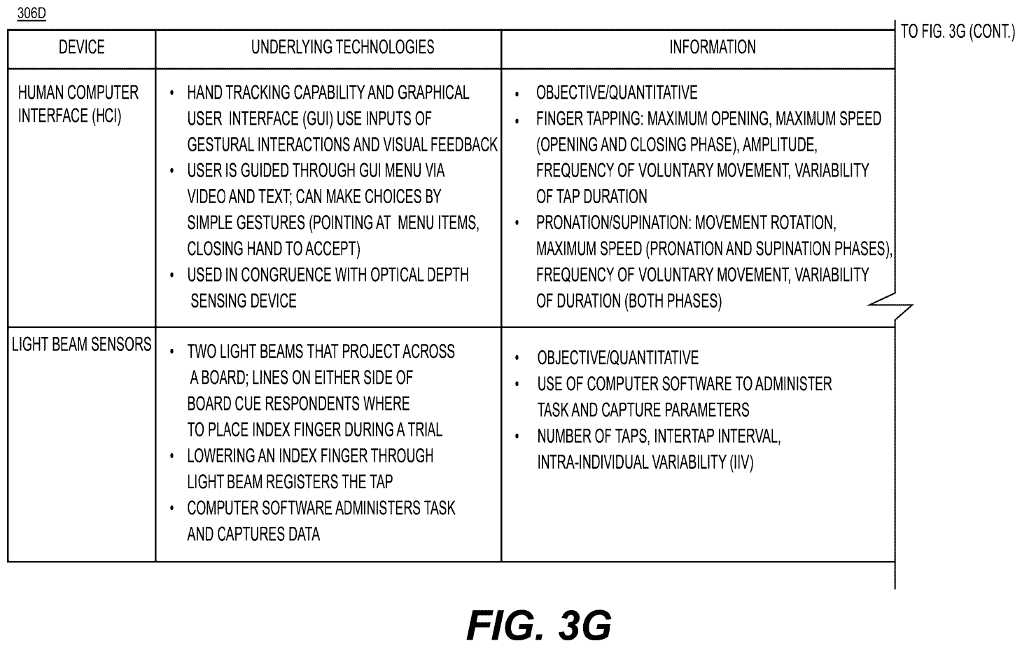

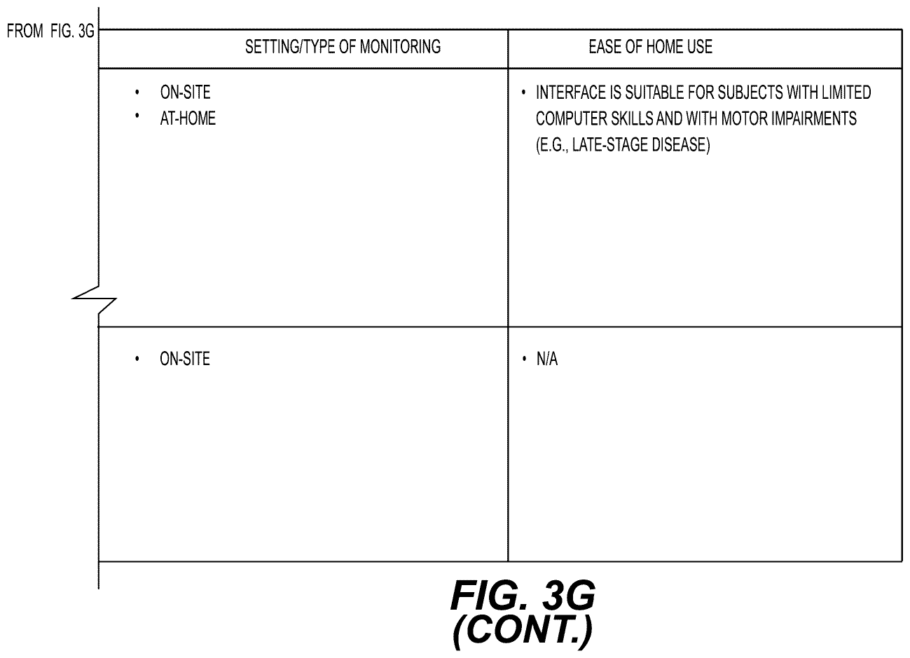

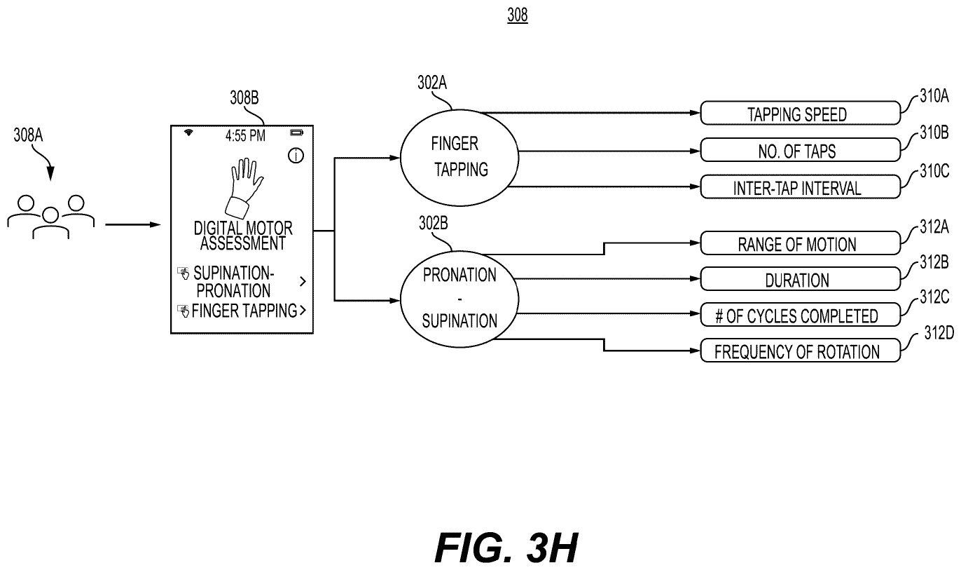

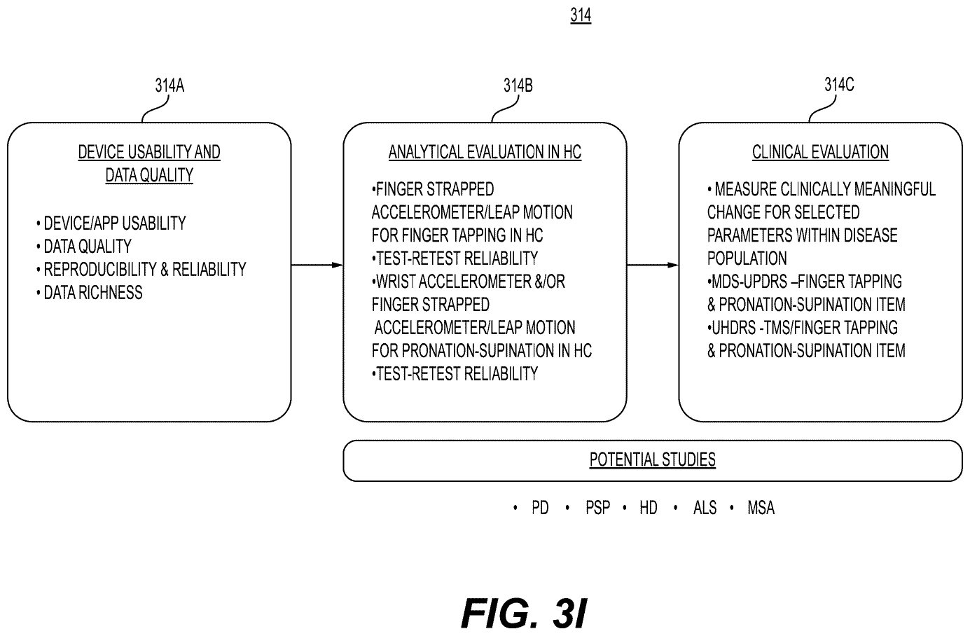

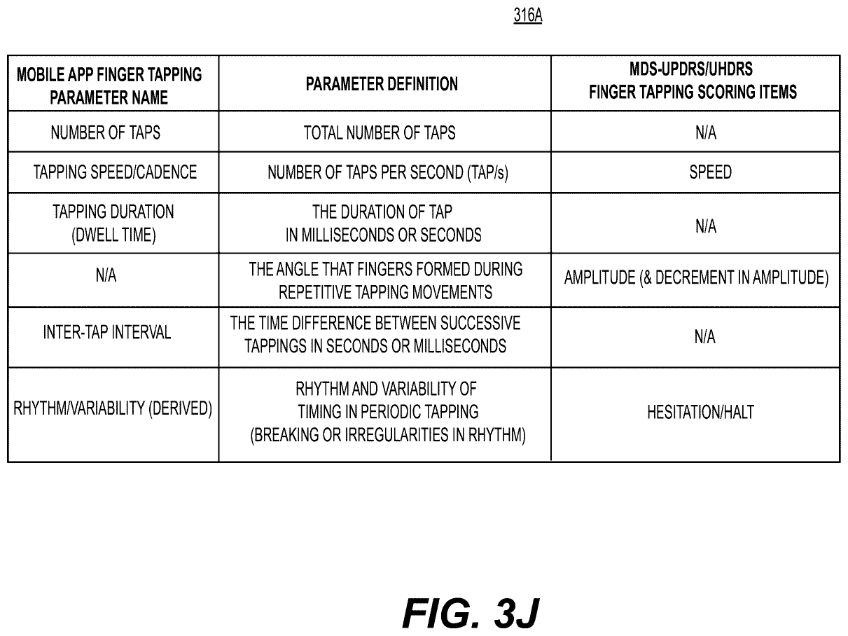

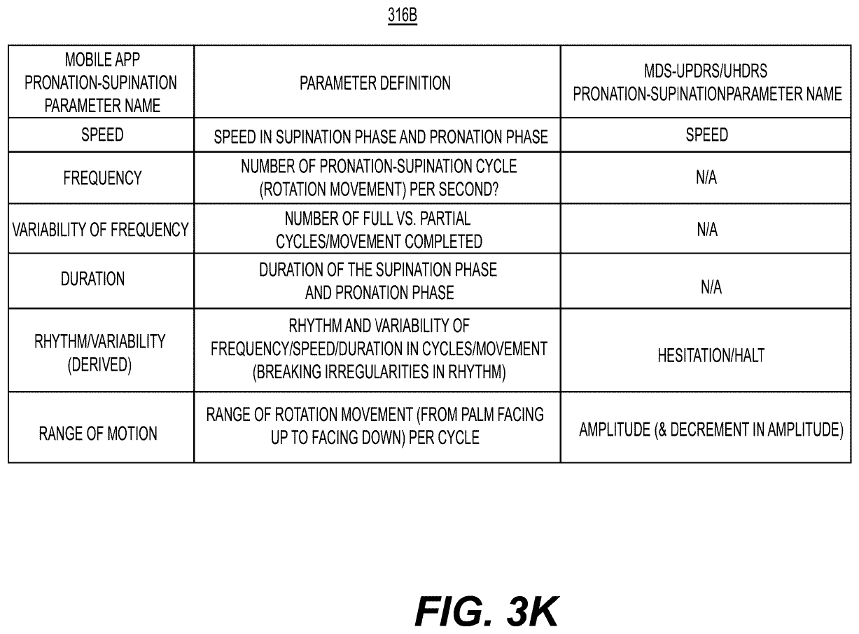

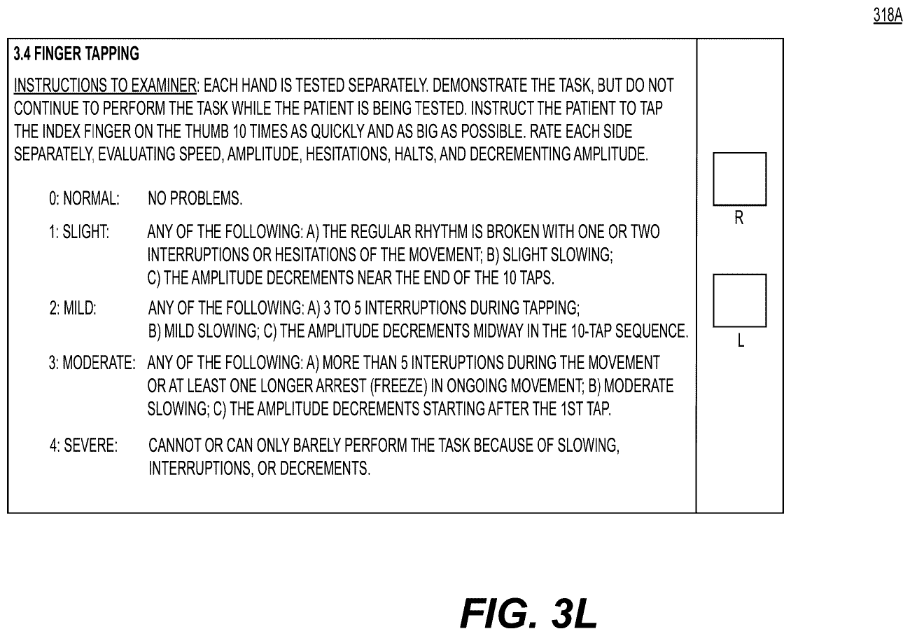

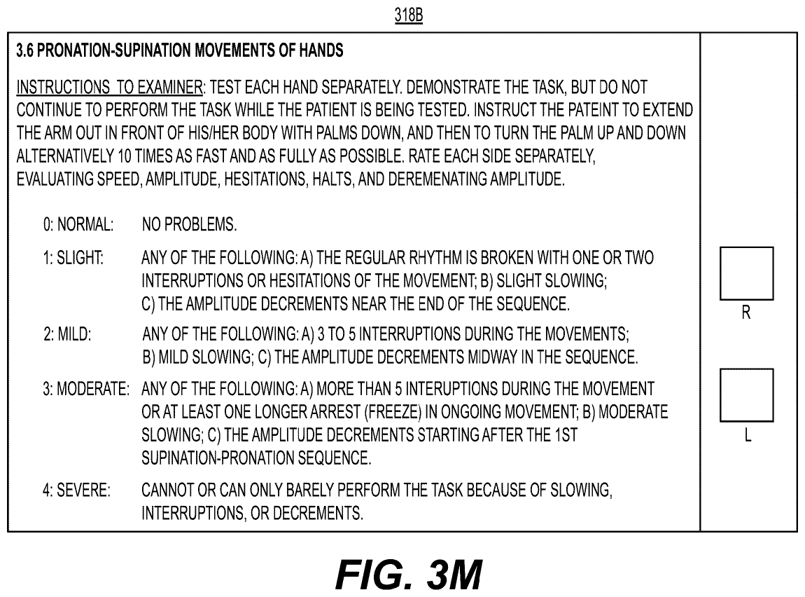

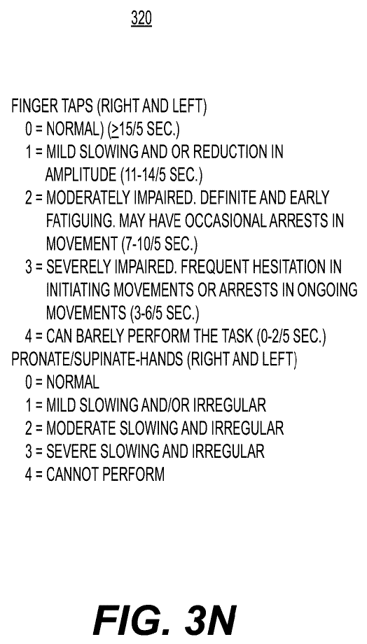

FIG. 3A is a flow diagram 300 for an experimental study conducted in accordance with aspects of the present disclosure and further discussed herein. At 310, users provide consent (e.g., via a software application using user device 102) to participate in the experimental motor assessment based study. At step 302A, a finger tapping assessments is performed and at step 302B, a rotation-based (e.g., proration and/or supination) assessments is performed. The assessments are repeated over time (e.g., two times after a one week duration). At 303, the users complete a mobile application usability survey. The study participation ends at 304.

The experimental study assesses the utility of a software application (e.g., mobile application) for the administration and recording of fine motor assessments for measuring neurodegenerative disease progression. The study is designed to understand user experience, perform initial EDA for finger tapping and rotation-based datasets, derive key descriptive statistics, develop descriptive models, and/or determine replicability and assess inter-relationships between different parameters.

Neurodegenerative diseases are often characterized by a limitation in fine motor function, which can occur as early symptoms before definitive diagnosis. Repeating movements of the hands, such as finger tapping and pronation/supination can be used in clinical practice to detect and measure bradykinesia. These movements are often incorporated as part of the standard clinical rating scales for diseases including Parkinson's disease, Huntington's disease, and Alzheimer's. The experimental study of FIG. 3A is implemented to assess the utility of a mobile application for the administration and recording of fine motor assessments to measure neurodegenerative disease progression (e.g., as predicted by a machine learning model output). The experimental study includes performing tasks that include a finger tapping test and a wrist rotation test, as discussed herein. Objectives of the experimental study include performing EDA on two task based datasets, deriving key descriptive statistics, generating descriptive models, determining replicability, and assessing inter-relationships between different parameters.

FIG. 3B is a table 302A for the finger tapping tasks and durations associated with the study of FIG. 3A, in accordance with aspects of the present disclosure. As shown, the finger tapping assessments performed at 302A includes various finger tapping tasks for respective durations. As shown at step 302B, the finger tapping tasks are implemented using a mobile device and respective interface. A first task is performed by alternatively tapping a first area of the interface using a right index finger and a second area of the interface using a right middle finger. A second task is performed by repeatedly tapping a third area of the interface using a right index finger. For the first task, users tap the targets with index and middle finger in an alternating pattern, tapping the buttons, as quickly as possible, until the buttons disappear after 15 seconds. Right-hand performance is tested first, followed by the left hand. After completion of index and middle finger tapping with both dominant and non-dominant hands, for the second task, users tap the buttons with index finger only, as quickly as possible, until the buttons disappear after 15 seconds. Two 15 second trials are performed per test, for each hand, twice (e.g., one week apart) for a total of eight tests.

FIG. 3C is a table 303A for rotation-based tasks and durations associated with the study of FIG. 3A, in accordance with aspects of the present disclosure. As shown, the rotation-based assessments performed at 303A includes various rotation tasks for respective durations. As shown at diagram 303B, the rotation-based tasks are implemented using a mobile device and respective interface. An example rotation task is performed by holding and rotating a mobile device in the user's left hand, as shown in diagram 303B. For this task, users are seated with their arms fully extended while holding the mobile device. The users then perform alternating hand rotation movements as fast and as fully as possible for 15 seconds. Two 15 second trials may be performed per test, for each hand, twice (e.g., one week apart) for a total of eight total tests.

The study of FIG. 3A includes 28 healthy patients that share similar socio-economic backgrounds (e.g., education, income, occupation). The study includes 21 females and 7 males ranging from 41-60 years of age. A majority of the participants share similar physical indicators which can reinforce expectation for limited sample differences.

As discussed herein, a mobile application may offer at-home and quantitative fine motor assessments. FIGS. 3D-3G include tables 306A-306D for device properties for motor assessment, in accordance with aspects of the present disclosure. Tables 306A-306D include various devices and their respective underlying technologies, information, setting and/or type of monitoring, and ease of home use information. As shown in table 306A, finger tapping/pronation-supination assessment using a mobile application is provided. The underlying technology related with such a mobile application include finger tapping on circles/target areas on a screen of a mobile device to capture tapping and force. It further includes holding a mobile device on a palm to rotate a wrist while having a straightened arm, as discussed herein. As also included in table 306A, the mobile application may be used on-site or in an at-home setting and may be easy to administer/use at home.

Table 306A includes information regarding a rating scale (e.g., for performance) and clinical reporting based assessments. Such assessments may be implemented by clinicians, may be subjective and/or semi quantitative, and may be performed on-site at a clinician site. Table 306A. Table 306A includes information regarding assessments using sensors attached to fingers and/or thumbs. Such assessments may include collecting sensed data during tapping and/or rotation, may include objective/quantitative analysis, may be performed on-site or at-home, and may be operationally challenging due to the positioning, attaching, and/or otherwise connecting the sensors.

Table 306B of FIG. 3E includes information regarding a neurophysiology laboratory manual tap board which may be a wooden board with a small lever attached to an analog counter. The tap board may provide objective/quantitative analysis for assessments that are performed on-site and not in a home setting. Table 306B also includes information regarding a QWERTY keyboard that includes switches and circuits to translate keystrokes for objective/quantitative analysis for assessments that are performed on-site or in a home setting. Table 306B includes information regarding a force transducer which may be transducer attached to a surface or a transducer attached to fingers. The force transducer may provide objective/quantitative analysis for assessments that are performed on-site and not in a home setting.

Table 306C of FIG. 3F includes information regarding a motion analyzer system which may include 2D and 3D image-based or passive marker-based analyzers that provide objective/quantitative analysis for assessments that are performed on-site and not in a home setting. Table 306C also includes information regarding goniometers that is used by placing a forearm in a pronated position with wrist, thumb, and fingers 2-4 supported by a brace while index finger taps. The goniometer may be used for objective/quantitative analysis for assessments that are performed on-site and not in a home setting. Table 306C also includes information regarding a mechanical keyboard and mechanical tools that include physical switches for parameter based analysis for assessments that are performed on-site and not in a home setting.

Table 306D of FIG. 3G includes information regarding a human computer interface (HCl) that includes hand tracking capability and GUI use inputs of gestural interactions and visual feedback for objective/quantitative analysis for assessments that are performed on-site or in a home setting such as for subjects with limited computer skills and motor impairments. Table 306D also includes information regarding light beam sensors that include light beams that project across a board for objective/quantitative analysis for assessments that are performed on-site and not in a home setting.

FIG. 3H is a flow diagram 308 for a digital multi-modal motor assessment (e.g., for neurodegenerative diseases) performed by one or more users 308A, in accordance with aspects of the present disclosure. As shown in FIG. 3H, a digital motor assessment 308B is implemented using a mobile application as discussed herein. The digital mobile assessment 308B includes the finger tapping motor assessment at step 302A and pronation/supination motor assessment at step 302B of FIG. 3A. The finger tapping motor assessment at step 302A produces signals related to tapping speed 310A, number of taps 310B, and inter-tap intervals 310C. The pronation/supination motor assessment at step 302B produces signals related to a range of motion 312A, duration 312B, number of cycles completed 312C, and a frequency of rotation 312D. An objective of the experimental study includes developing a digital endpoint that can indicate disease progression. Such a digital end point may be based on correlation with disease stages/states and with clinically meaningful changes. The digital endpoint may include a potential for more efficient proof of concept (POC) trials with larger effect sizes, more frequent assessment (e.g., less variation), and/or uncovering early stage changes that may not be otherwise evident.

FIG. 3I is a flow diagram 314 for evaluations using motor assessment (e.g., for neurodegenerative diseases), in accordance with aspects of the present disclosure. Step 314A describes device usability and data quality based on test device and/or software application usability, data quality, reproducibility and reliability, and data richness. Step 314B describes analytical evaluation in health controls (HC) (e.g., healthy participants) which may be conducted using a finger strapped accelerometer/leap motion for finger tapping in HC, a wrist accelerometer and/or finger strapped accelerometer/leap motion for pronation/supination motor assessment in HC. A respective test-retest reliability for each evaluation may be determined. Step 314C describes a clinical evaluation for measuring clinically meaningful changes for selected parameters within disease populations. For MDS-UPDRS, finger tapping and pronation/supination motor assessments may be performed. For Unified Huntington's Disease Rating Scale (UPHRS), transcranial magnetic stimulation (TMS)/finger tapping and pronation/supination motor assessments may be performed. Evaluations using motor assessments may be performed for various conditions such as PD, progressive supranuclear palsy (PSP), ALS, multiple system atrophy (MSA), and/or the like.

FIG. 3J is a table 316A for finger tapping scoring, in accordance with aspects of the present disclosure. Table 316A shows parameters of interest for a finger tapping assessment including number of taps, tapping speed/cadence, tapping duration (dwell time), finger angles, inter-tap intervals, rhythm/variability and related parameter definitions and MDS-UPDRS/UHDRS finger tapping scoring items.

FIG. 3K is a table 316B for rotation scoring, in accordance with aspects of the present disclosure. Table 316B shows parameters of interest for a pronation/supination assessment including speed, frequency, variability of frequency, duration, rhythm/variability, range of motion, and related parameter definitions and MDS-UPDRS/UHDRS pronation/supination names.

FIG. 3L is an example instruction 318A for a finger tapping assessment, in accordance with aspects of the present disclosure. The instruction 318A is provided as supplement to the experimental study of flow diagram 300 and includes instructions to an examiner for demonstrating a task, instructing a participant, and rating the performance based on the provided scale from 0-4.

FIG. 3M is an example instruction 318B for a rotation assessment, in accordance with aspects of the present disclosure. The instruction 318B is provided as supplement to the experimental study of flow diagram 300 and includes instructions to an examiner for demonstrating a task, instructing a participant, and rating the performance based on the provided scale from 0-4.

FIG. 3N shows example scoring 320 for motor assessment, in accordance with aspects of the present disclosure. As shown, scoring 320 includes scoring based on a scale (e.g., 0-4) ranging from normal operation (e.g., 0) to not being able to perform a task (e.g., 4).

The results from the instruction 318A, instruction 318B, and the scoring 320 are compared against results output by mobile application based assessments (e.g., as described in reference to FIG. 3A). The comparison may be used to validate performance of the mobile application based assessments.

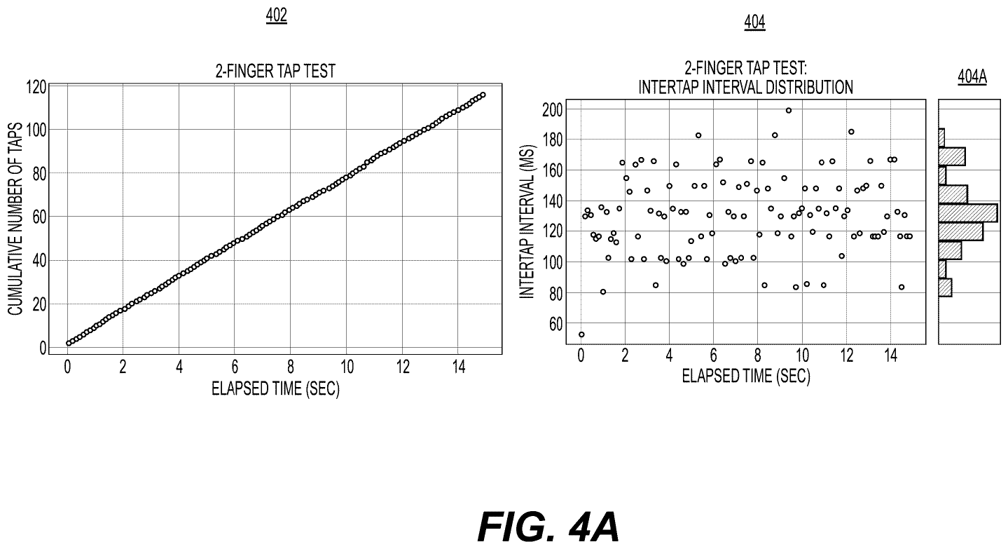

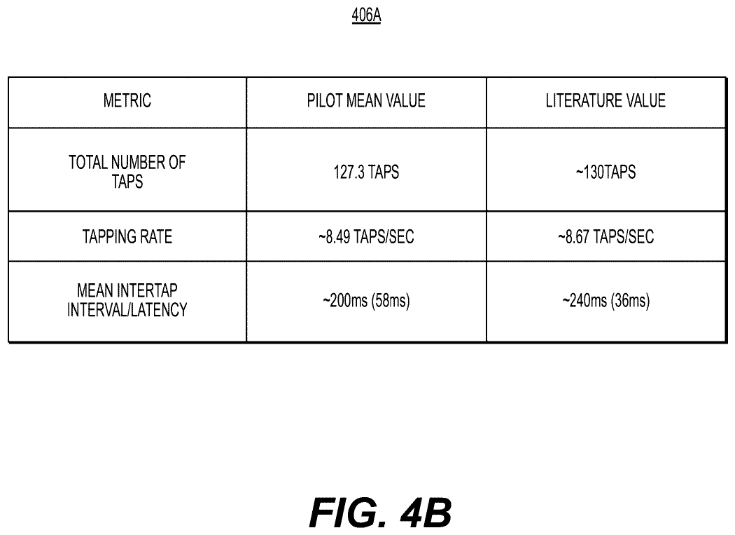

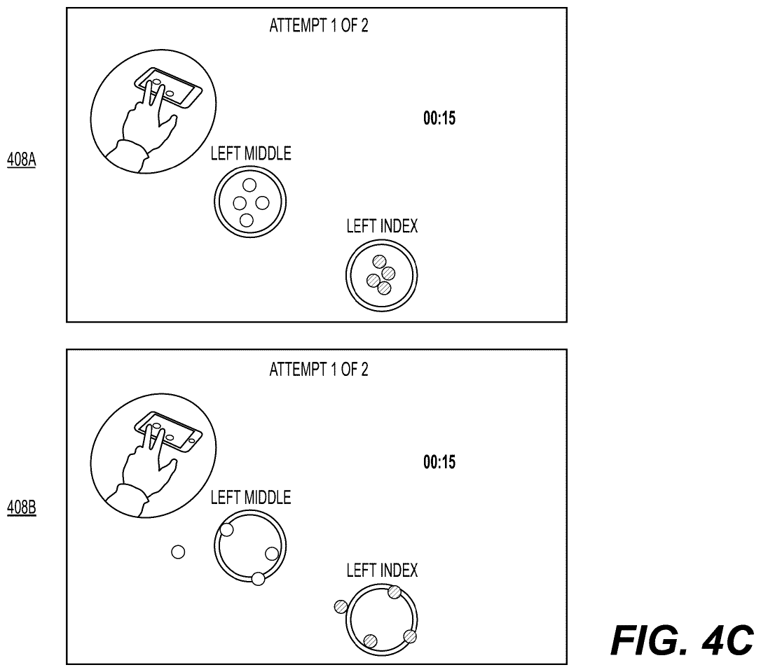

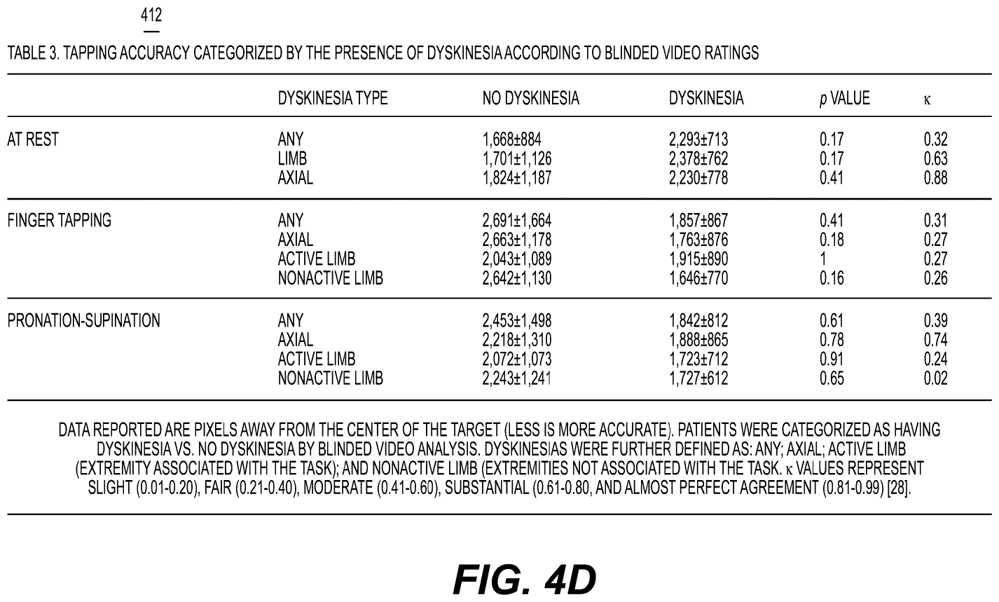

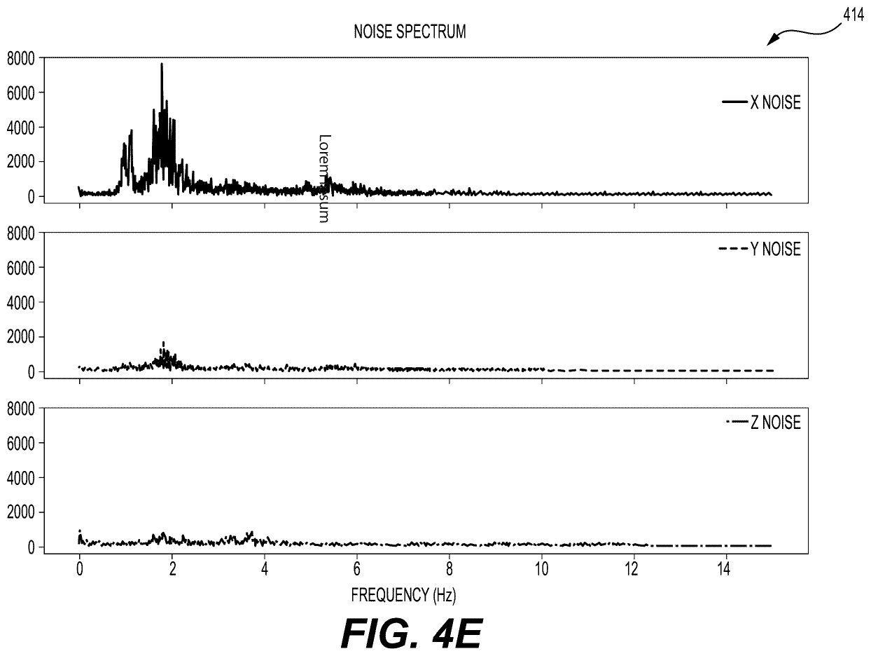

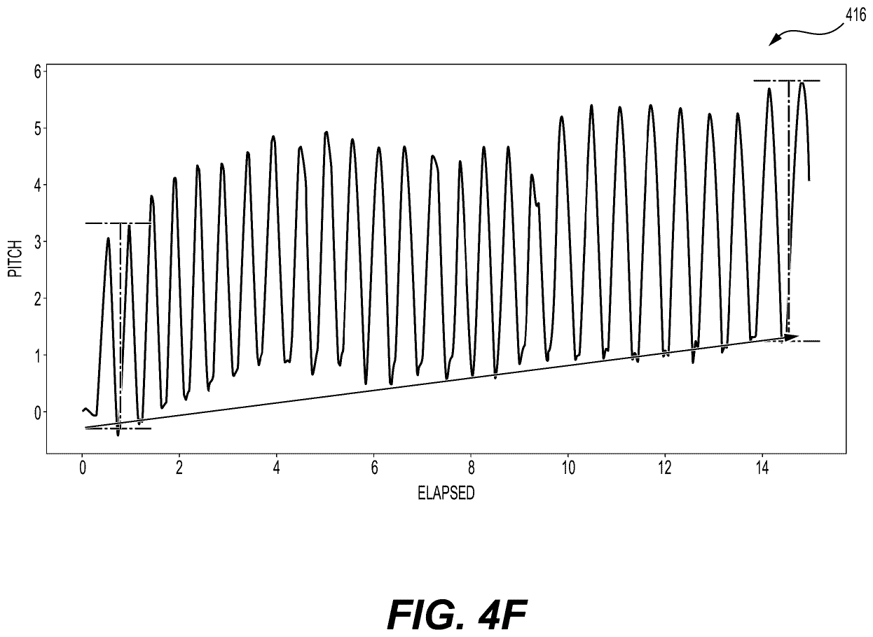

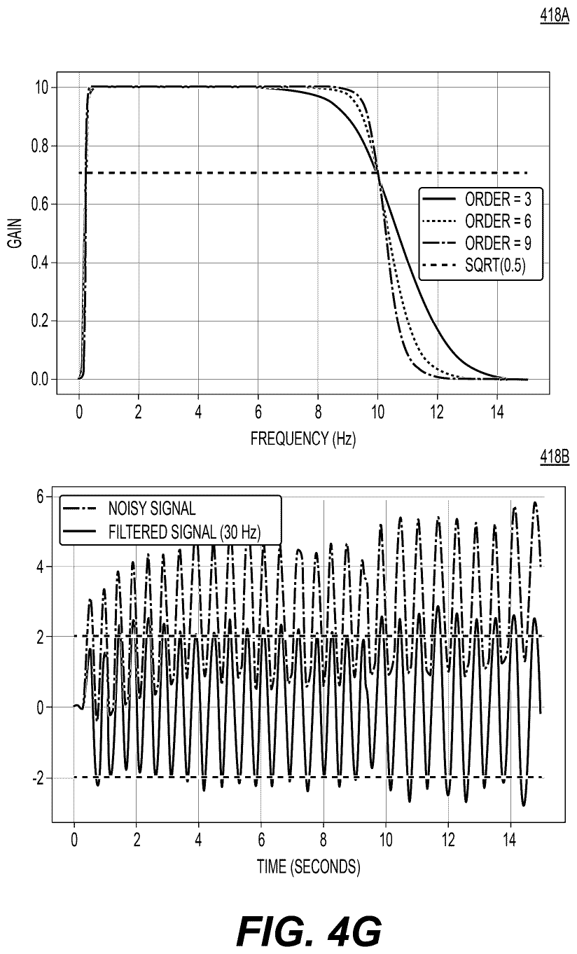

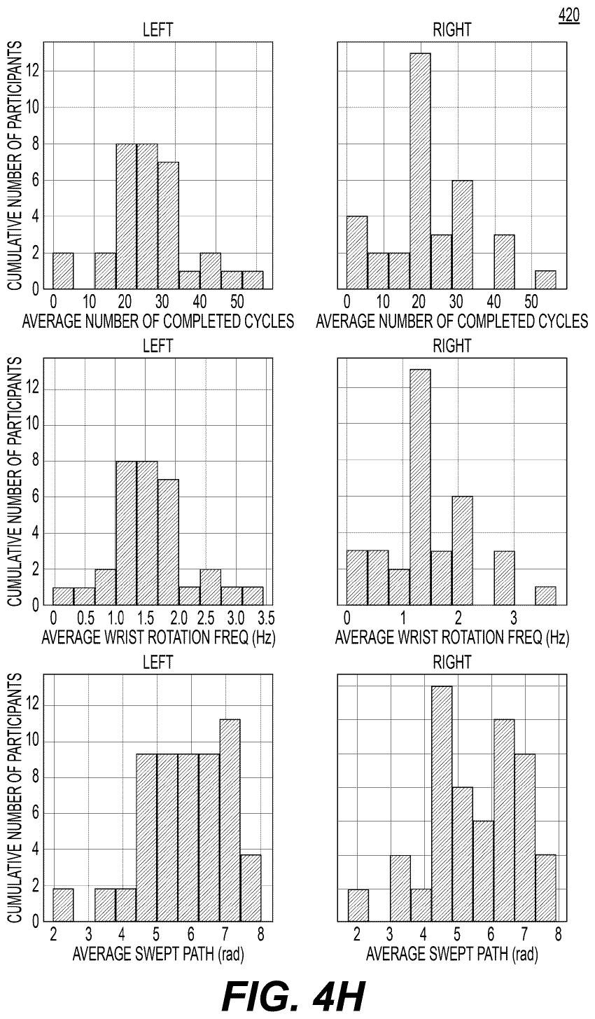

FIGS. 4A-4B show example results for motor assessments, in accordance with aspects of the present disclosure. The example results shown in FIGS. 4A-4B indicate that the data generated based on the finger tapping assessment is consistent with standard literature data, validating the mobile application based finger tapping assessments. FIG. 4A includes chart 402 that shows results of a two finger tap test. As shown in chart 402, a cumulative number of taps is plotted against an elapsed duration of the test (e.g., approximately 15 seconds). As shown, taps are expected to increase monotonically with time, producing an approximate linear trend. Deviations from the shown linear trend may be indicative of events which may be used for medical condition predictions. FIG. 4B shows chart 404 that shows inter-tap interval distribution for the two finger tap test. As shown in chart 404, inter-tap intervals measured in milliseconds are plotted against an elapsed duration of the test (e.g., approximately 15 seconds). As shown, distribution of inter-tap interval is Gaussian and ranges from approximately 50 ms to approximately 200 ms. Area 404A shows a histogram of inter-tap distributions based on ranges of milliseconds.

FIG. 4B shows a table 406A that includes data measured during the experimental study in comparison to standard values found in applicable literature. As shown, observed number of total taps, tapping rate, and mean inter-tap interval/latency values are compared to standard values. The observed values indicate that the mobile application based data is comparable in reliability to standard literature results.

A summary of significant results for PD mean standard deviation and HC mean standard deviation, as well as respective p-values, may be determined as disclosed in Mitsi et al. Biometric Digital Health technology for Measuring Motor Function in Parkinson's Disease: results from a Feasibility and Patient satisfaction study. Front. Neurol. 8:273 (2017), which is incorporated by reference herein. The variables measured may include, for example, two-target total taps, two-target tapping velocity, two-target average interval, single target total taps, single target tapping average interval, reaction time, and reaction accuracy.

Speed measured as degrees per second may be compared to a speed coefficient of variance (CV) for HC, PSP, PD, and MSA conditions as disclosed in Luft et al. Deficits in tapping accuracy and variability in tremor patients. Journal of NeuroEngineering and Rehabilitation 16:54 (2019), which is incorporated by reference herein. The speed measured as degrees per second for HC may be greater than the corresponding speed measured as degrees per second for PSP, PD, and MSA patients. The speed CV for HC may be lower than the speed CV for PSP, PD, and MSA patients.

Total finger taps may be compared to finger tapping latency for health older adults (HOA) in comparison to participants with Addison's disease (AD), Mild cognitive impairment (MCI), and PD, as disclosed in Roalf et al. Quantitative assessment of finger tapping characteristics in mild cognitive impairment, Alzheimer's disease, and Parkinson's disease J Neurol. 265(6): 1365-1375 (2018), which is incorporated by reference herein. The total finger taps may be significantly different between HOAs and total finger taps exhibited by participants with AD, MCI, and PD. The finger taping latency may be significantly different between HOAs and participants with AD and MCI, though not significantly different between HOAs and participants with PD. Such information may be used by a prediction machine learning model to, for example, predict diseases onset.

FIG. 4C shows diagrams 408A and 408B for a finger tapping assessments in a manner similar to that discussed in FIG. 3B, in accordance with aspects of the present disclosure. Diagrams 408A and 408B may be generated based on respective users using a digital screen to record tap position relative to left middle and left index targets to perform respective finger tapping assessments. The finger tapping assessments may allow for direct measurement of tapping accuracy (e.g., how close to the target the user taps) and tapping variability (e.g., how consistent multiple taps by the same user are). The corresponding measurements are used to quantify the severity of bradykinesia and/or dyskinesia. As shown in diagram 408A, an example first user's distribution of finger taps may be contained within a respective left middle target region and a respective left index target region. As shown in diagram 408B, an example second user's distribution of finger taps may not be contained within a respective left middle target region and a respective left index target region. Accordingly, prediction machine learning model may predict that diagram 408A corresponds to a healthy user and diagram 408B may correspond to a user exhibiting dyskinesia, based on corresponding signals generated based on the assessments associated with diagrams 408A and 408B.

According to embodiments, healthy patients may show low tapping variability, substantially tapping within the respective target region, as is consistent with standard literature. Two-finger tapping tests are expected to receive a reduction in middle finger degree of freedom due to an enslaving effect of the index finger where the middle finger is mechanically coupled to and restricted by the index finger. The degree of enslavement may be an additional disease indicator used by a prediction machine learning model to predict disease onset, outcomes, and/or trends. Mean distance from a target center (e.g., a pixel) for two finger tapping may be determined and/or plotted. Mean distance from a target center for index finger tapping may be determined and/or plotted. Mean distance from a target center for middle finger tapping may be determined and/or plotted. In an example, results, mean distance for middle finger tapping is more accurate than both the two finger tapping and index finger tapping. Accelerometer data for tapping accuracy for a 2 Hz tapping task may be determined and/or charted. HC deviation may be lower than the deviation for users with an essential tremor (ET) and users with PD.

FIG. 4D is a table 412 showing tapping accuracy categorized by the presence of dyskinesia, in accordance with aspects of the present disclosure. A prediction machine learning model may use the data provided in table 412 to determine a cluster for a user and/or to make predictions (e.g., disease onset). As shown in table 412, patients without dyskinesia exhibit ratings at rest, bas on a finger tapping assessment, and based on a pronation and supination assessment when compared to patients with dyskinesia.

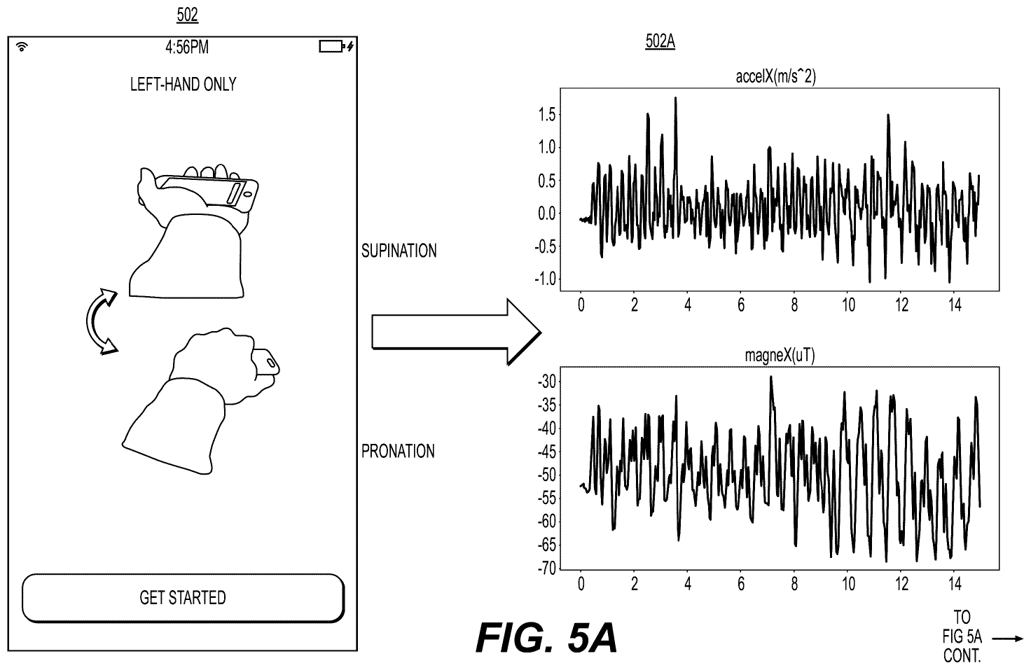

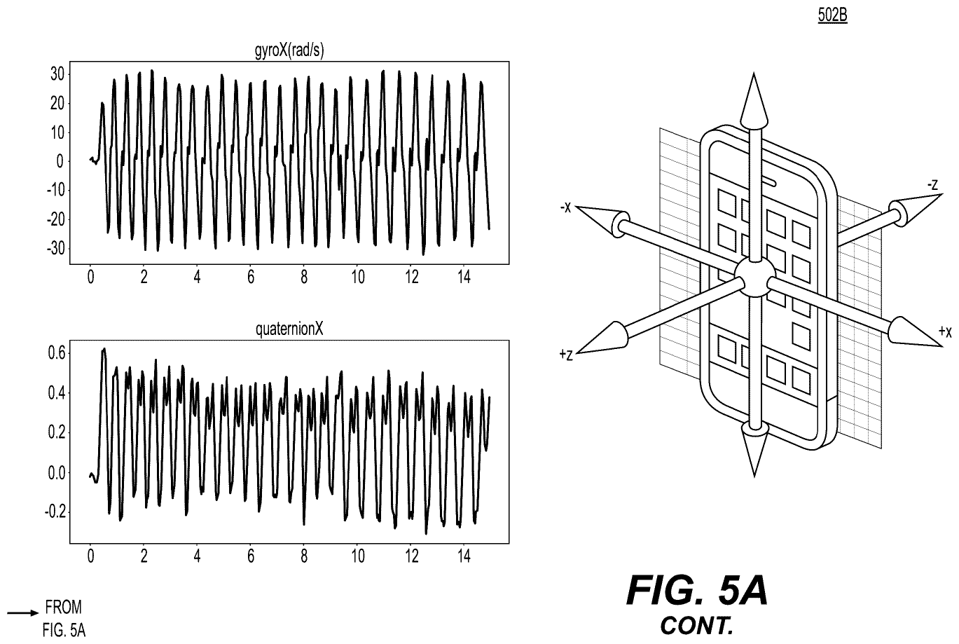

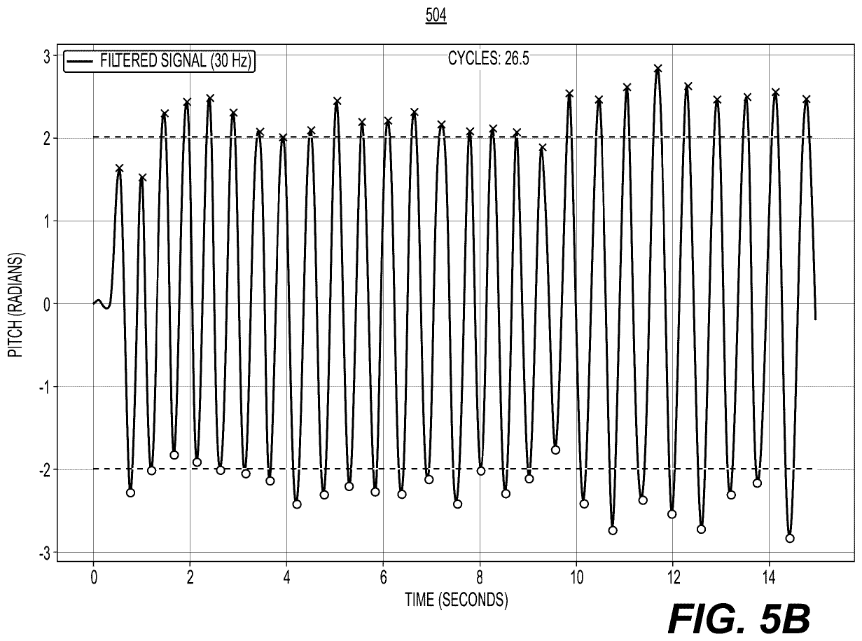

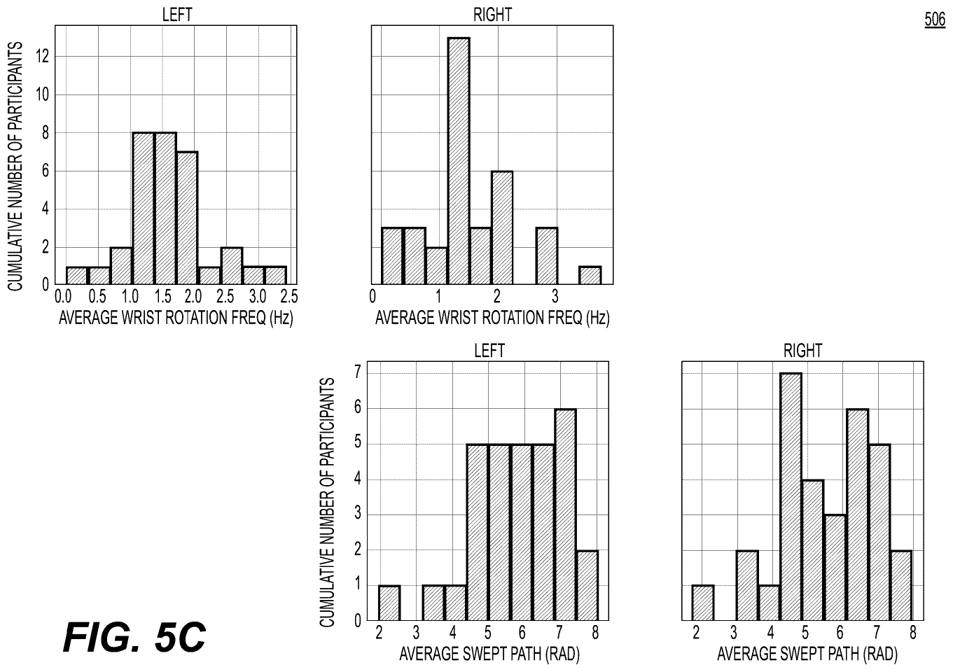

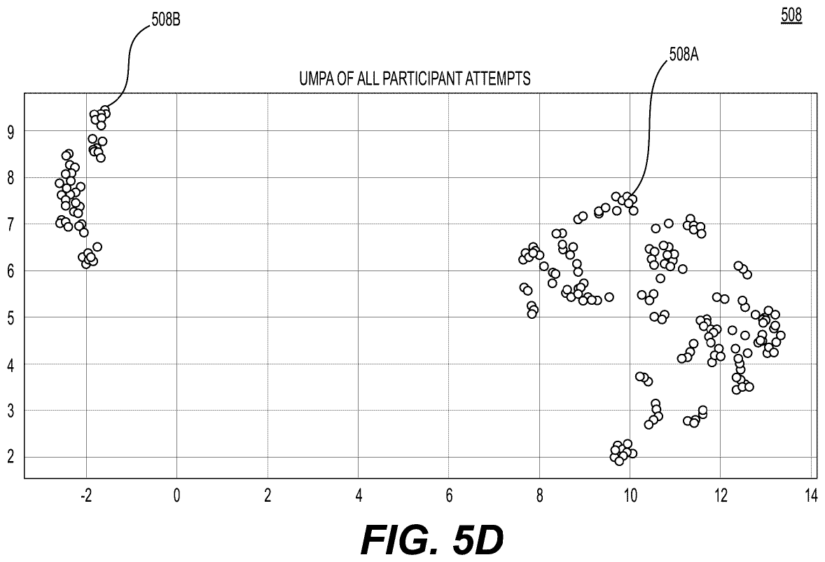

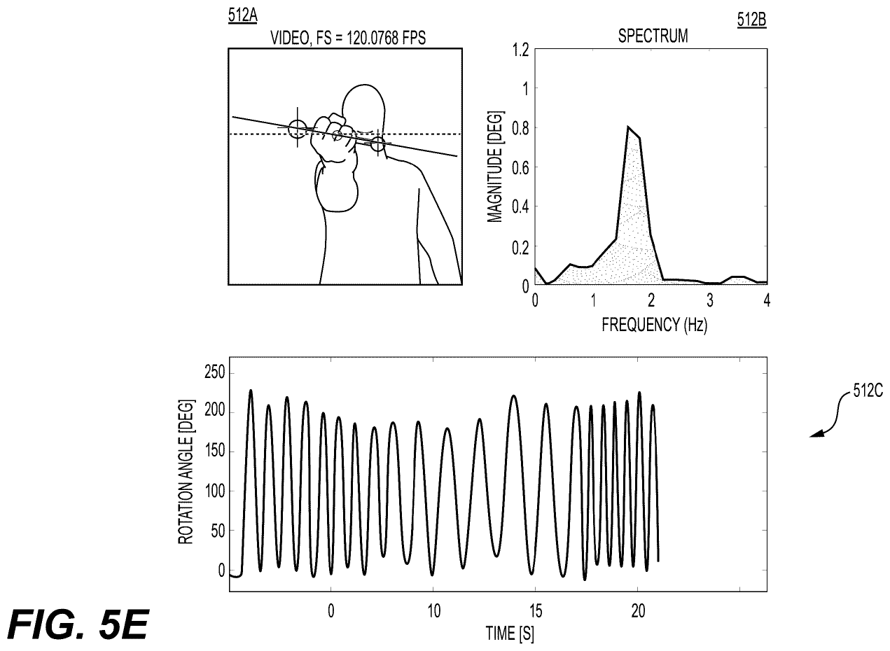

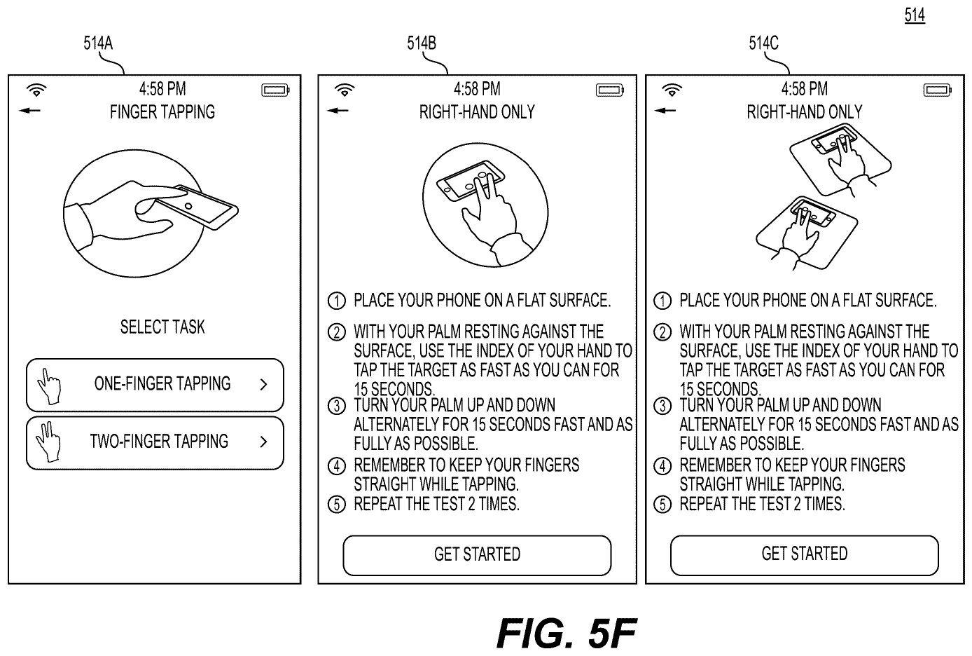

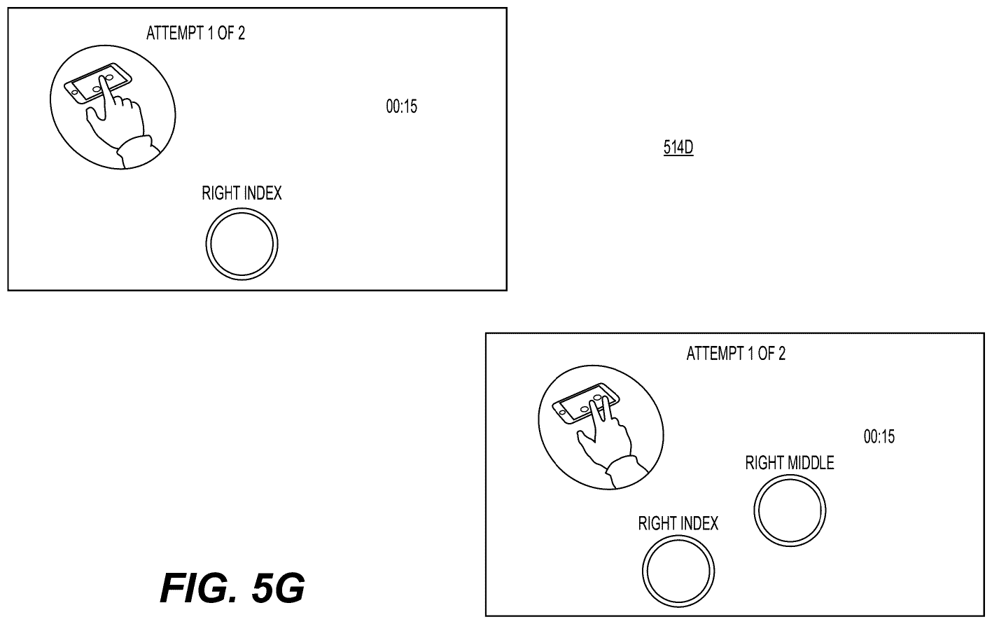

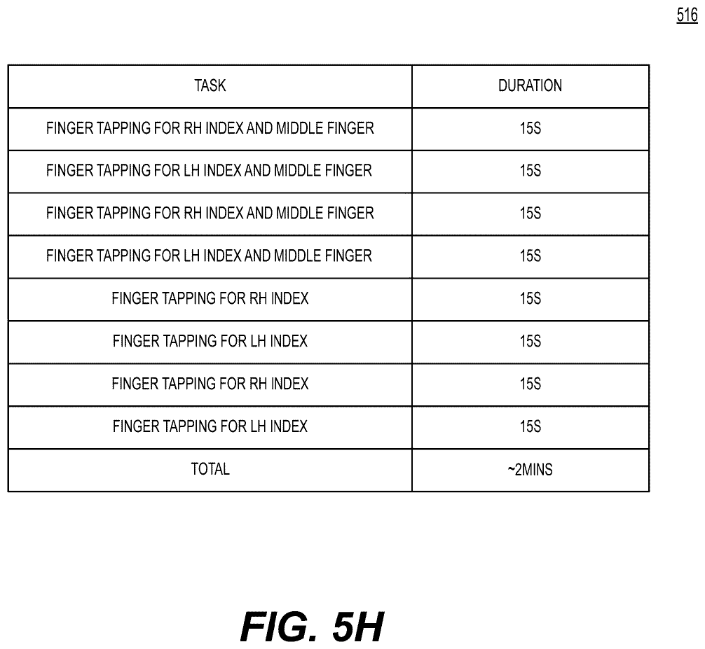

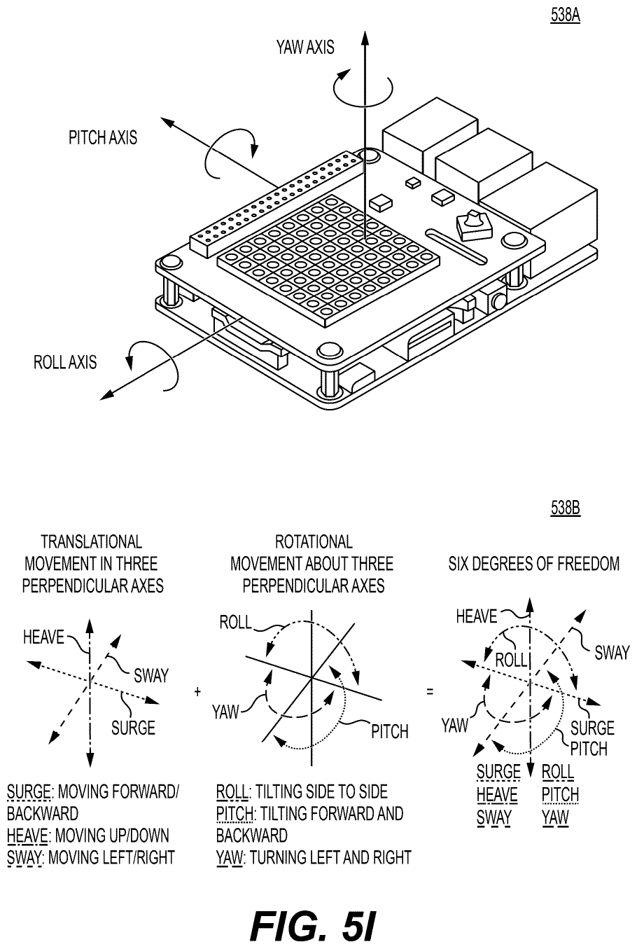

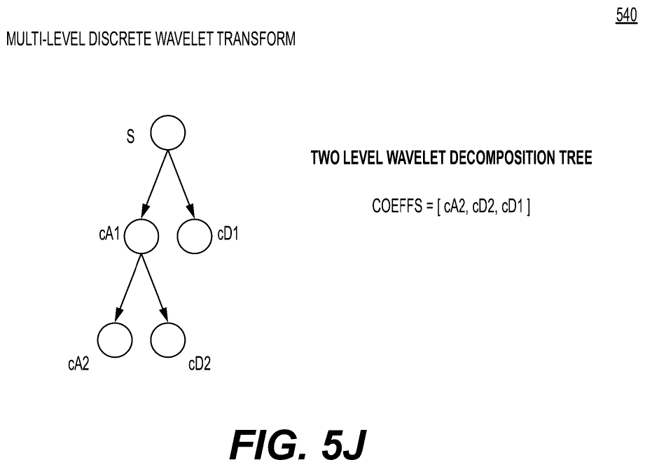

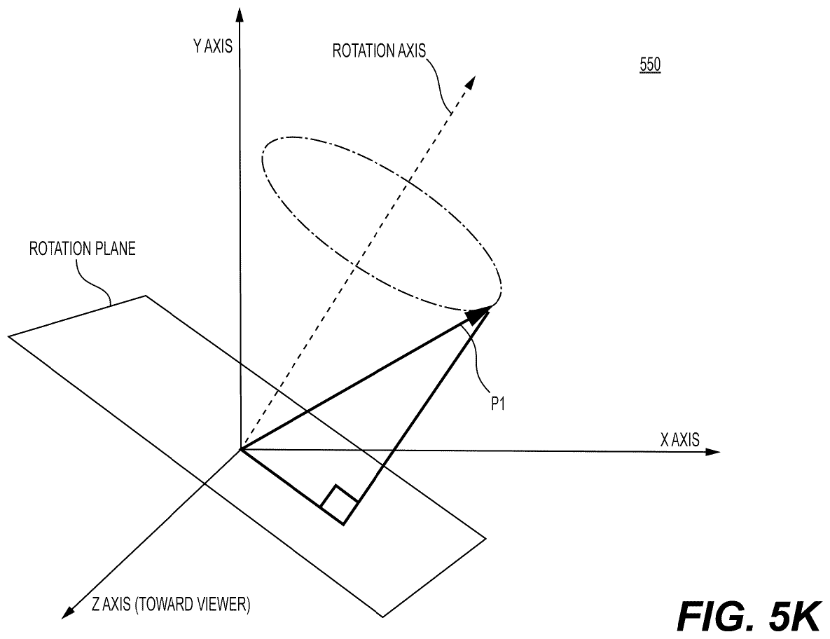

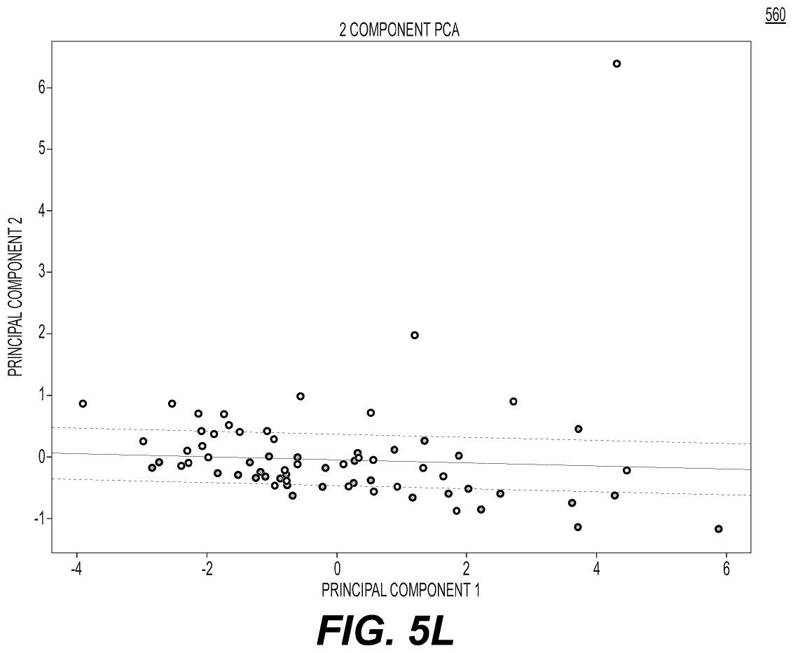

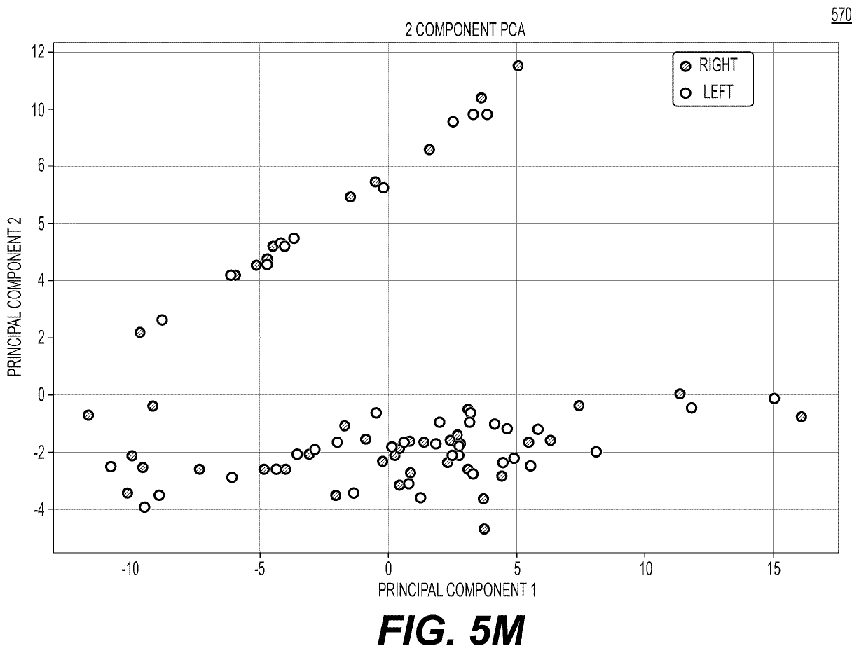

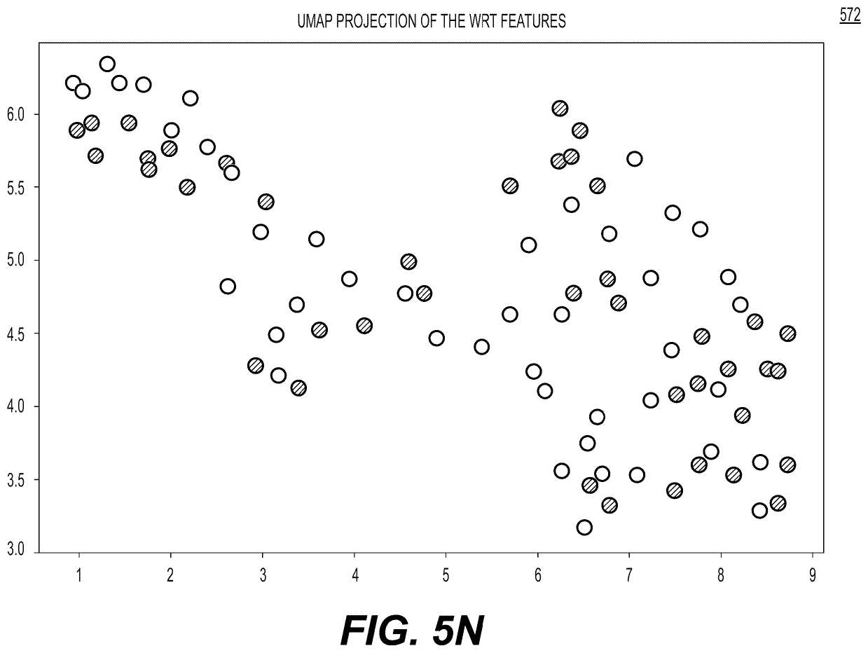

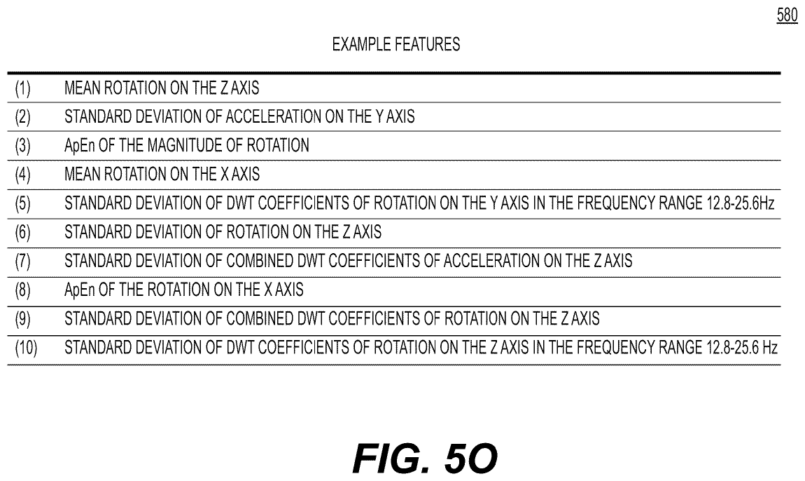

FIGS. 4E-4G show charts for noise reduction for motor assessment, in accordance with aspects of the present disclosure. Sinusoidal signals output as a result of a rotation-based assessment (e.g., sinusoidal signals 502A further discussed herein in reference to FIG. 5A) are processed at noise reduction component 106A. Sensor noise generates drift and/or bias that is removed by noise reduction component 106A to generate transformed signals for accurate measurement. FIG. 4E shows charts 414 exhibiting a noise spectrum for X, Y, and Z noise and FIG. 4F shows chart 416 exhibiting noise drift in pitch over elapsed time. Gyroscopes show both very low and very high frequency noise. Such noise is inherent to internal measurement units (IMUs) to some degree and is generally considered to be uncorrelated across multiple sensors. Such noise poses a hurdle to orientation calculation, as discussed herein. Integration for determining instantaneous sensor measurements compounds the error into random bias and sensor drift. Noise reduction component 106A normalizes such noise, based on multiple sensor and/or orientation data.

FIG. 4G shows chart 418A and 418B for sensor fusion and/or signal filtering based attenuation of noise. Bandpass filtering, as shown in chart 418A, can Bandpass filtering can help attenuate the noise, but the ripple in the passband can affect signal gain (introducing a different kind of bias). Butterworth filters aim to have a flat passband, but some ripple may remain. Sensor fusion, as shown in chart 418B can aid in reducing the noise, in accordance with the techniques disclosed herein. As shown in chart 418B, a noisy signal maybe filtered to remove noise, resulting in a filtered signal that does not exhibit sensor drift. Sensor fusion solutions may be system specific and may be based device and/or sensor components. Noise reduction component 106A may homogenize signals from multiple sensors which may ease downstream processing burden.

FIG. 4H shows charts 420 generated as a result of a rotation-based assessment, in accordance with aspects of the present disclosure. As shown in charts 420 for left and right hand results, health patients show consistent rotational frequency. Specifically, as shown in charts 420, participants exhibited a relatively consistent rotational frequency at approximately 1.5 Hz. Corresponding cycles completed were consistent at approximately 23 cycles over the course of 15 seconds. The swept path averages at approximately 5.6 rad (approximately 320 degrees) which corresponds to approximately two times the expected range of motion. Additional filtering (e.g., noise cancellation), adjustments, and/or validation may be applied to confirm reliability of the results shown in charts 420.

FIGS. 5A-5D show results from a rotation-based motor assessment, in accordance with aspects of the present disclosure. The rotation-based motor assessment may include performing pronation and/or supination movements as discussed herein. The rotation-based motor assessment may generate sinusoidal signals (e.g., regular sinusoidal signals) and may include irregular high frequency components. As shown in FIG. 5A, pronation and supination movements 502 are performed during the rotation-based assessment. Sinusoidal signals 502A are generated based on the pronation and supination movements 502. Sinusoidal signals 502A are generated based one or more sensors and/or coordinates such an accelerometer, gyroscope, magnetometer, and/or quaternion quadrants. As discussed herein, the different sinusoidal signals may be compared to each other to account for sensor drift and/or sensor fusion. Noise cancelling may be implemented to generate the transformed signal 504 of FIG. 5B based on the different sinusoidal signals 502A. The transformed signal 504 may be used to determine movement information based on an absolute zero coordinate 502B. Based on the rotation-based motor assessment, progressive reduction in movement properties (e.g., amplitude, speed, etc., and/or a combination thereof) may indicate presence or onset of neurodegenerative disease. Such presence or onset may not be adequately assessed by traditional clinical scales and clinicians may not detect subtle and/or mixed changes in properties such as amplitude, accuracy, velocity, and/or rhythm of movement.

The post-processed transformed signal 504 includes orientation information related to swept path, number of cycles completed, average rotational speed and/or the like. The orientation information is used to obtain clinically relevant metrics such as cycles per test, total range of motion (e.g., swept path), rotational frequency, changes in range of motion across and within tests, changes in rotational frequency across and within tests, etc. Such metrics are compared to standard literature values to validate the techniques disclosed herein.

FIG. 5C shows charts 506 including left and right hand based results from the pronation and supination assessment in references to FIGS. 5A and 5B. As shown, participants exhibited a relatively consistent rotational frequency at approximately 1.5 Hz. Corresponding cycles completed were consistent at approximately 23 cycles over the course of 15 seconds. The swept path averages at approximately 5.6 rad (approximately 320 degrees) which corresponds to approximately two times the expected range of motion. Additional filtering (e.g., noise cancellation), adjustments, and/or validation may be applied to confirm reliability of the results shown in charts 506.

FIG. 5D shows a Uniform Manifold Approximation and Projection (UMAP) 508 generated based on the pronation and supination assessment discussed above. UMAP 508 is based on all participants and all events for the participants, such that each data point shown on UMAP 508 represents a single participant. UMAP 508 shows, for example, at least two clusters which may be generated by user cluster module 108A of FIG. 1A. A first user data point 508A may be associated with a first cluster whereas a second user data point 508B may be associated with a second cluster. Each respective cluster may be used by prediction module 108B of machine learning model 108 to make predictions about a given user based on the user's respective cluster. Data from the pronation and supination assessment may be z-scored across all trials for a given feature. UMAP 508 is shown without application of a disease cohort, though addition of a disease cohort may provide additional feature refinement.

FIG. 5E shows an example rotation motor assessment 512A and corresponding real time features extracted based on the rotation motor assessment 512A, in accordance with aspects of the present disclosure. Spectrum 512B is generated based on the corresponding motor data associated with the rotation motor assessment 512A and is updated based on changes to the motor data as the user performs rotation motor assessment 512A. Spectrum 512B shows the magnitude, in degrees, plotted against the frequency of motion. Chart 512C is generated based on the corresponding motor data associated with the rotation motor assessment 512A and is updated based on changes to the motor data as the user performs rotation motor assessment 512A. Chart 512C shows the rotation angle, in degrees, over time as the user performs rotation motor assessment 512A. As discussed herein, due to uncertainties involved with sensor drift and integration, a high-quality reference is used to validate metrics, filters, and/or sensor fusion algorithms. Orthogonal measures that do not suffer from the same bias may be used for the validation and/or correction. High-speed motion capture, as shown in FIG. 5E may be used as a POC for validation. Such a high-speed capture may allow direct measurement of wrist orientation, without the need to integrate further. Instantaneous measures are determined through differentiation.

Results from the experimental study show that 85% of participants found the software application easy to download, navigate, and were willing to use the application more frequently (e.g., weekly) in future studies, a home. Results further showed that 78% of participants found the application easy to use, 81% were satisfied with the application, and 48% favored weekly assessments. Results further showed that 15% of participants did not find the instructions helpful, 63% favored the application interface, 7% experienced application malfunctions, and 7% reported frustration with the application. Results further showed that 93% of participants were satisfied by both the finger tapping and rotation-based assessments and 7% marked the rotation-based task as least favored.

FIG. 5F is a flow diagram 514 for a finger tapping motor assessment, in accordance with aspects of the present disclosure. Flow diagram 514 may be provided to a user prior to or while completing a finger tapping task. Interface 514A provides finger tapping task options for the finger tapping motor assessment. Interface 514B and interface 514C provide instructions for a two-finger tapping task upon selection of a two-finger tapping task from interface 514A and include a button to begin the corresponding task.

FIG. 5G is a diagram 514D for a finger tapping motor assessment, in accordance with aspects of the present disclosure. Diagram 514D provides interfaces provided to a user during performance of a respective task. The interfaces include a single target area for a single finger task and two target areas for a two-finger task. The interfaces of diagram 514D may be provided in accordance with the task criteria shown in table 516 of FIG. 5H.

FIG. 5I shows a rotation test device 538A and associated movements shown at diagram 538B, in accordance with aspects of the present disclosure. Rotation test device 538A may be used for rotation-based assessments, as disclosed herein. As shown in FIG. 5I, rotation test device 538A may be used to generate, for example, six degrees of freedom based on a roll axis, pitch axis, and yaw axis. The three axes may be used to determine transitional movement in three perpendicular axes and may also be used to determine rotational movement about three perpendicular axes. Given the more complex nature of the IMU waveforms, higher order time series analyses are used to reduce each attempt down to a given number of features (e.g., approximately 130 features; approximately 10 feature from each of approximately 13 signal channels corresponding to the 13 degrees of freedom discussed herein). Channel level variation and asymmetry are measure three statistical moments including mean, standard deviation, and skewness. Irregularities in motion are quantified through per channel approximate entropy. Higher entropy is more irregular. Standard parameters for window size and ratio of signal standard deviation are used (m=2, r=0.2). Multi-resolution analysis is carried out via 3-level Discrete Wavelet Decomposition as shown in the example decision tree 540 of FIG. 5J using a two level wavelet decomposition tree. Daubechies 10th order wavelet is used for decomposition. Such a decomposition provides insight into the importance and characteristics of the various frequency sub-bands within each signal. Mean and standard deviations are measured for three levels of sub-bands including approximately 15-30 Hz, approximately 7.5-15 Hz, and a combination of approximately 0-3.85 Hz, approximately 3.85 Hz-7.5 Hz, and approximately 7.5-15 Hz. Sub-bands are based on the sampling frequency (approximately 30 Hz) and the Nyquist limit (approximately 15 Hz) chosen to give full coverage across the entire frequency range. Signal wide aggregate measures include a number of cycles completed, a number of full cycles, and a number of partial cycles

FIG. 5K shows a graphical representation 550 of raw data for a rotation-based motor assessment, in accordance with aspects of the present disclosure. The graphical representation 550 corresponds to raw data collected based on a rotation-based assessment. One or more position sensors may be used to determine a rotation plane, rotation axis, and projection P1 based on rotation-based movements. Raw data corresponding to graphical representation 550 is collected using one or more sensors at a frequency of approximately 30 Hz. Time is given in millisecond (ms) and may be represented as a Unix epoch time. An Euler 3D rigid-body measures acceleration (m/s2) for X, Y, and Z axes, rotation (radians/s) for X, Y, and Z axes. A hyper-complex three dimensional rigid-body measures four dimensional quaternions for X, Y, Z, and W axes. Magnetic measurements (microT) are collected in three dimensions along X, Y, and Z axes. Data including metadata (e.g., universal unique identifiers for test, phone model, operating system, etc.) may also be collected.

FIG. 5L shows an example principal component analysis (PCA) plot 560, in accordance with aspects of the present disclosure. The PCA plot 560 is derived based on per user features. As shown in PCA plot 560, study data provides proof of concept that patient aggregate features can be used for clustering and separation. A hyperplane can be drawn to separate the left and right hand based information. Separation between dominant and non-dominant hands may be determined using techniques similar to those used to generate PCA plot 560. As participants in the experimental study have similar demographics (e.g., age, gender, education, and occupational background), the tight clustering exhibited in PCA plot 560 is validated. Greater separation may be identified using a robust algorithm (e.g. decision tree, random forest, etc.), as discussed herein.

FIG. 5M shows an example PCA plot 570 including intermingled right and left hand data, in accordance with aspects of the present disclosure. FIG. 5N shows an example UMAP plot 572 (projection plot) corresponding to PCA plot 570 of FIG. 5M, in accordance with aspects of the present disclosure. Both the PCA plot 570 and UMAP plot 572 show that right and left hands are intermingled as, for healthy participants, separation is not expected. The experimental study data provides proof of concept that patient aggregate features can be used for clustering. As participants in the experimental study have similar demographics (e.g., age, gender, education, and occupational background), the tight clustering exhibited in PCA plot 570 and UMAP plot 572 is validated.

FIG. 5O shows an example feature table 580, in accordance with aspects of the present disclosure. Feature table 580 includes example extracted features, as discussed herein. The example features are based on mean, standard deviation, and like values. Features in feature table 580 may be extracted by a feature extraction machine learning model and may be used to determine predictions by a prediction machine learning model, as discussed herein.

The motor assessments and/or results discussed in reference to FIGS. 3A-5O may be used to determine a clinical outcome. The clinical outcome may include one or more of identifying biomarkers, screening patient populations, identification of inclusion criteria, identification of disease progression, identification of a treatment, predicting medical conditions/disease progression/treatment effects, and/or the like. Alternatively, or in addition, as discussed herein, motor assessments and/or results discussed in reference to FIGS. 3A-5O may trigger a subsequent action. The trigger action may include one or more of generating an updated motor assessment, triggering a repeat motor assessment, outputting a treatment, implementing a treatment, modifying a database, and/or the like.

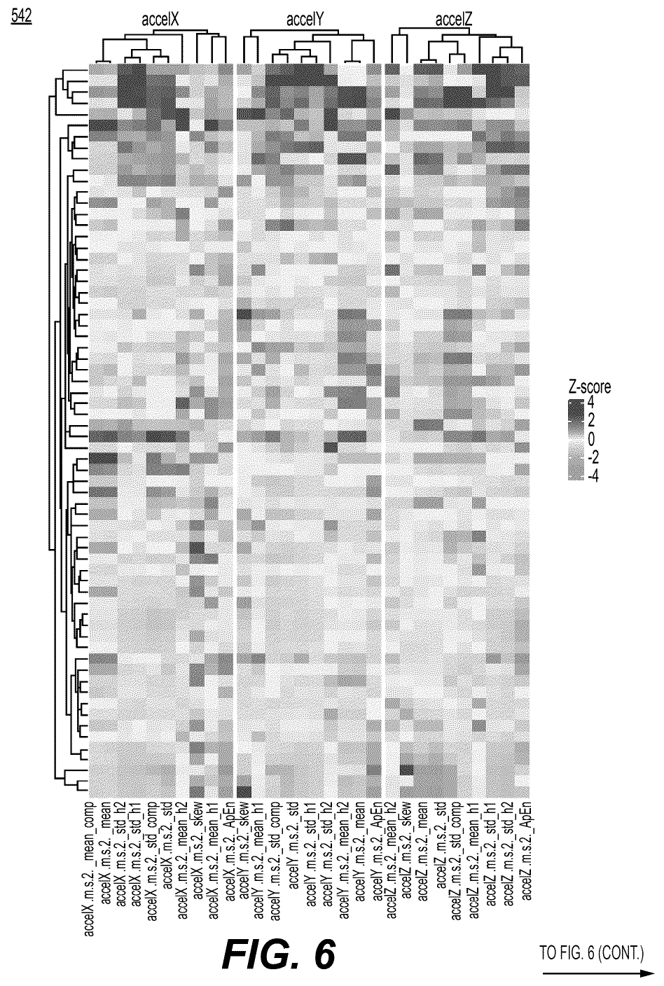

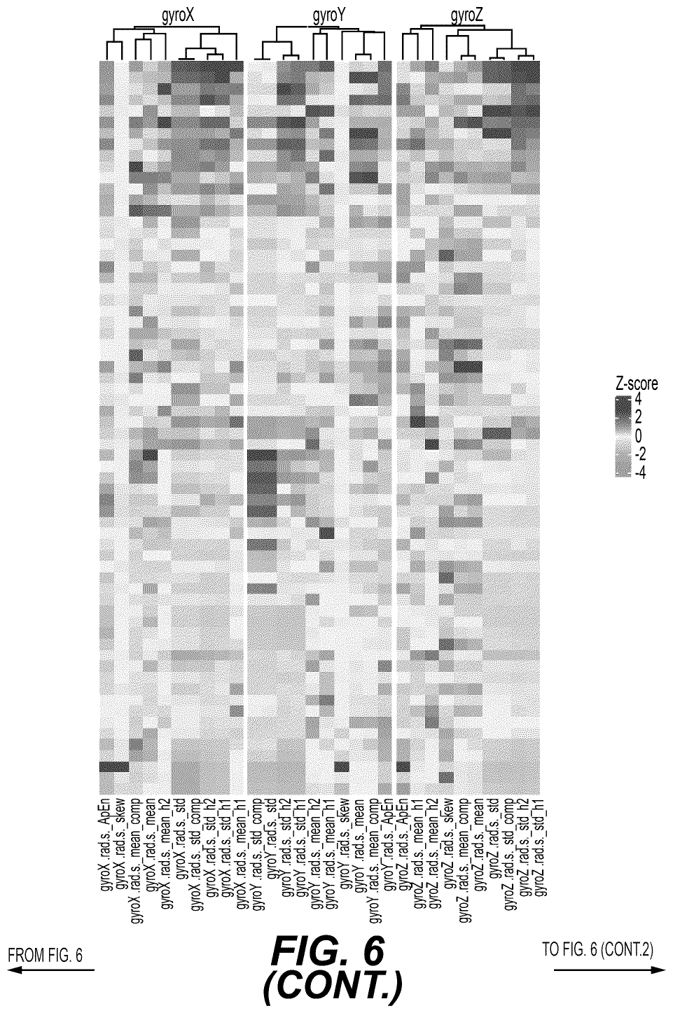

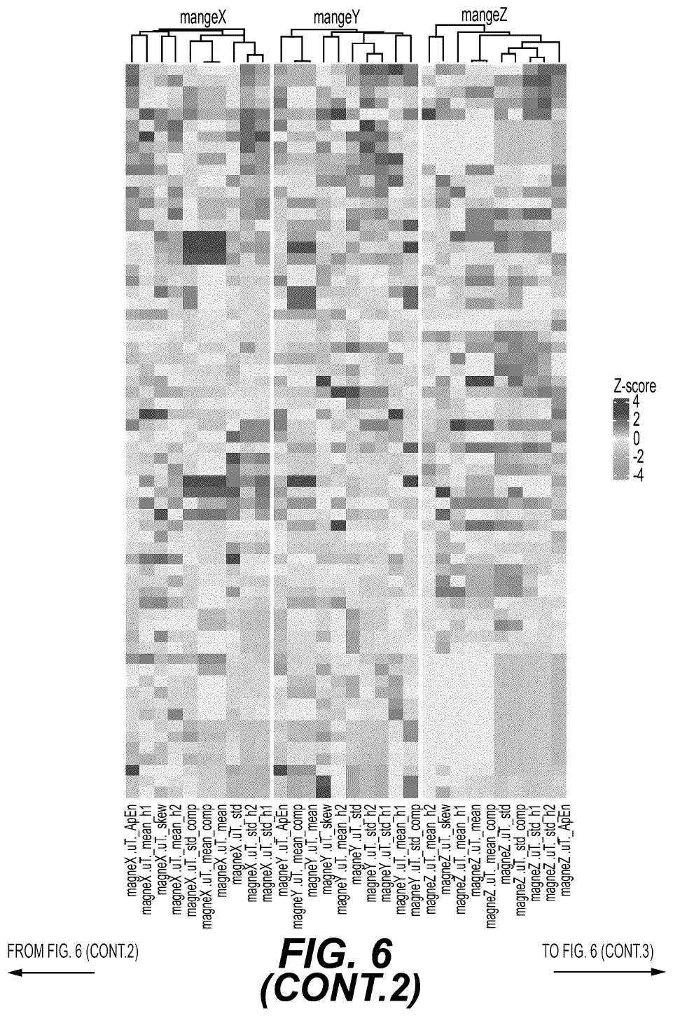

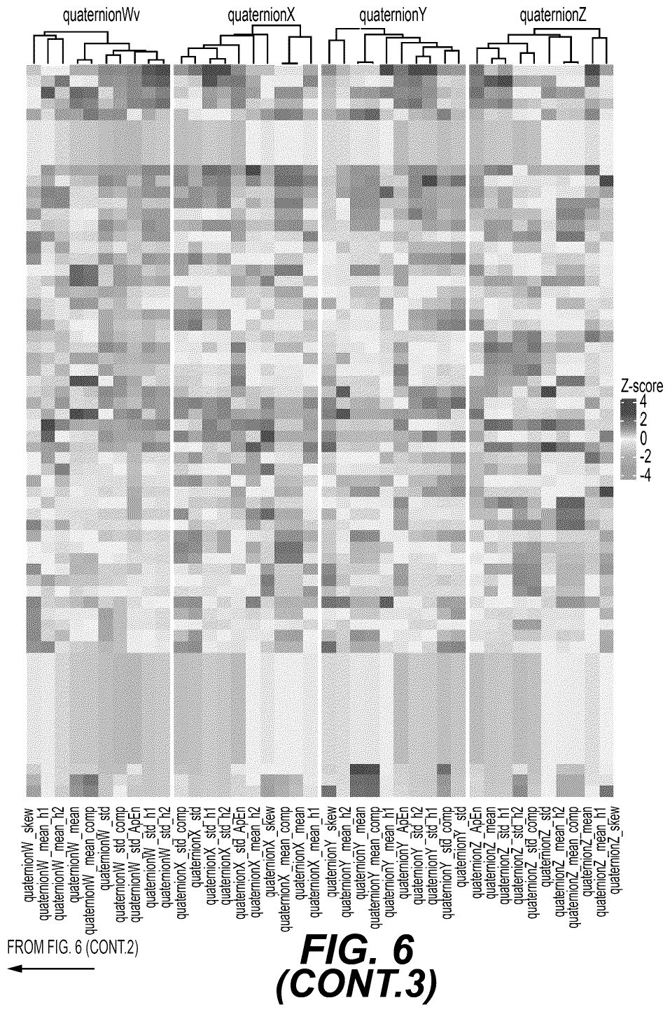

FIG. 6 shows a z-score chart 542 for motor assessment, in accordance with aspects of the present disclosure. Z-score chart 542 is generated based on all participants for all attempts (e.g., 584 attempts). The feature data extracted in accordance with the techniques disclosed herein is z-scored across all trails within a feature. Z-score chart 542 provides clear variability within many of the features. Z-score chart 542 is generated without specificity based on disease cohorts. Even without disease cohort, clear clustering within some of the features is provided via z-score chart 542. Addition of disease cohorts may allow for additional feature refinement. Example extracted features are shown via z-score chart 542 (bottom). As discussed herein, such a z-score chart and/or the associated data may be generated for a group of individuals that perform the motor assessments discussed herein. Such z-score charts and/or underlying data may be used to identify key biomarkers associated with clinical outcomes. Such key biomarkers may be used to determine inclusion and/or exclusion criteria for clinical trials, for treatment recommendations and/or administration, for generating graphical interfaces ordered based on the underlying data, and/or the like.

Finger tapping based disease progression results may be determined accordance with aspects of the present disclosure. Finger tapping based sensor features may be used by a prediction machine learning model to output predictions. Such features may be used to output predictions during an early stage of disease progression. Such features may be correlated with corresponding clinical items of MDS-UPDRS. Participants with PD may be differentiated based on Hoehn and Yahr stage 1 vs state 2. Corresponding results may be provided in view of one or more of a confidence interval, an intra-class correlation coefficient, based on a less affected side, and/or based on a more affected side. Both less affected (L) and more effected (M) sides may exhibit feature differences in an expected direction.

MDS-UPDRS finger tapping for a more effected side and MDS-UPDRS finger tapping for a less effected side may be determined. Tapping speed variability for the respective MDS-UPDRS finger tapping may be output (e.g., via a plot or other output).

Rotation-based disease progression results may be determined, in accordance with aspects of the present disclosure. Rotation-based sensor features may be used by a prediction machine learning model to output predictions. Such features may be used to output predictions during an early stage of disease progression. Such features may be correlated with corresponding clinical items of MDS-UPDRS. Corresponding results may be provided in view of one or more of a confidence interval, an intra-class correlation coefficient, based on a less affected side, and/or based on a more affected side. Both less affected (L) and more effected (M) sides may exhibit feature differences in an expected direction.

MDS-UPDRS rotation results for a more effected side and MDS-UPDRS rotation results for a less effected side may be determined. Hand turning speed variability for the respective MDS-UPDRS pronation and supination may be output (e.g., via a plot or other output).

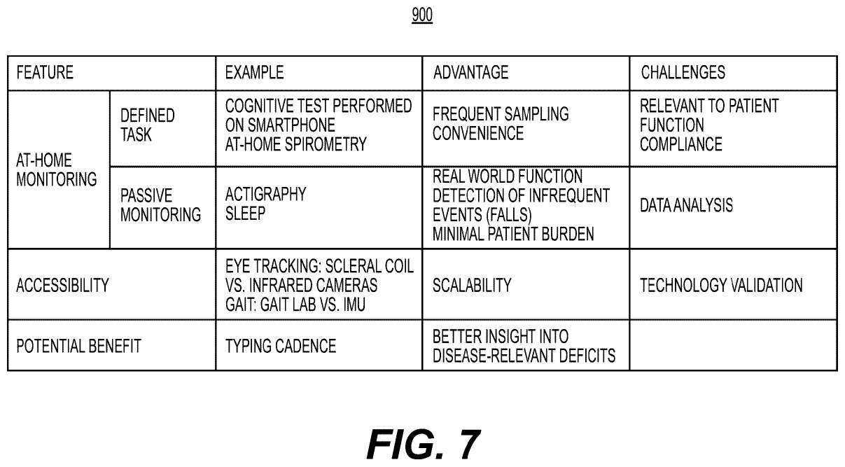

FIG. 7 is table 900 including motor assessment benefits, in accordance with aspects of the present disclosure. As shown, potential advantages of the digital motor assessments disclosed herein may include at home monitoring for defined tasks and/or passive monitoring, accessibility, and other potential benefits. Table 900 includes example features, advantages, and challenges associated with digital motor assessment.

As disclosed herein, one or more implementations disclosed herein may be applied by using a machine learning model. As recited herein, “a machine learning framework” may include one or more machine learning models. According to implementations disclosed herein, a first output of a first machine learning model may be provided to a second machine learning model such that the second machine learning model may output a second output. Although a first and second machine learning model are provided as examples, it will be understood that embodiments disclosed herein are not limited to two machine learning models and any applicable number of machine learning models may be used to implement the techniques disclosed herein.

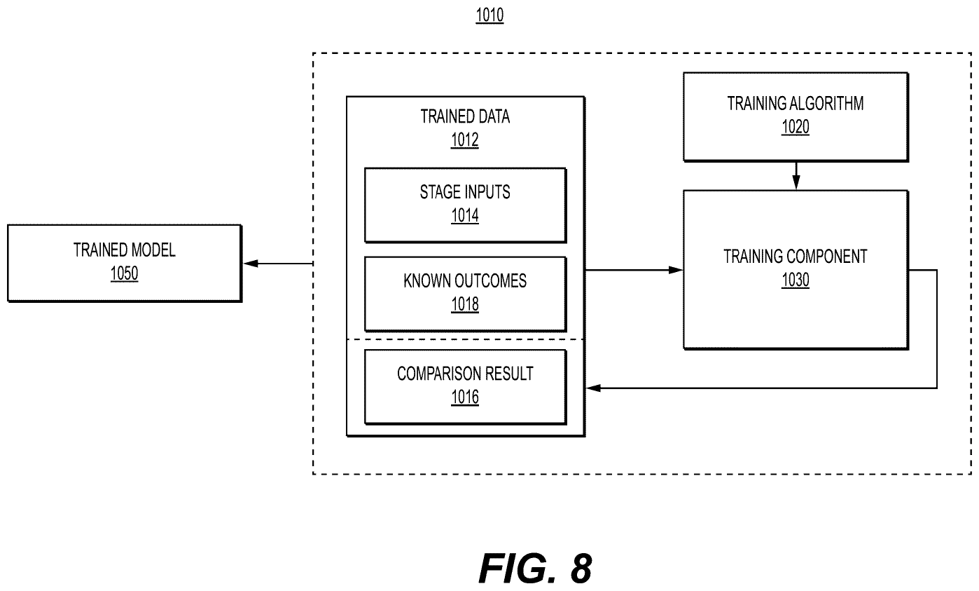

A machine learning model as disclosed herein may be trained using one or more components or steps of FIGS. 1-7. As shown in flow diagram 1010 of FIG. 8, training data 1012 may include one or more of stage inputs 1014 and known outcomes 1018 related to a machine learning model to be trained. The stage inputs 1014 may be from any applicable source including a component or set shown in the figures provided herein. The known outcomes 1018 may be included for machine learning models generated based on supervised or semi-supervised training. An unsupervised machine learning model might not be trained using known outcomes 1018. Known outcomes 1018 may include known or desired outputs for future inputs similar to or in the same category as stage inputs 1014 that do not have corresponding known outputs.

The training data 1012 and a training algorithm 1020 may be provided to a training component 1030 that may apply the training data 1012 to the training algorithm 1020 to generate a trained machine learning model 1050. According to an implementation, the training component 1030 may be provided comparison results 1016 that compare a previous output of the corresponding machine learning model to apply the previous result to re-train the machine learning model. The comparison results 1016 may be used by the training component 1030 to update the corresponding machine learning model. The training algorithm 1020 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of the flow diagram 1010 may be a trained machine learning model 1050.

A machine learning model disclosed herein may be trained by adjusting one or more weights, layers, and/or biases during a training phase. During the training phase, historical or simulated data may be provided as inputs to the model. The model may adjust one or more of its weights, layers, and/or biases based on such historical or simulated information. The adjusted weights, layers, and/or biases may be configured in a production version of the machine learning model (e.g., a trained model) based on the training. Once trained, the machine learning model may output machine learning model outputs in accordance with the subject matter disclosed herein. According to an implementation, one or more machine learning models disclosed herein may continuously update outputs based on feedback associated with use or implementation of the machine learning model outputs.

Djurić-Jovičic\acute{ } et al. Finger tapping analysis inpatients with Parkinson's disease and atypical parkinsonism. Journal of Clinical Neuroscience 30 49-55 (2016), Luft et al. Distinct cortical activity patterns in Parkinson's disease and essential tremor during a bimanual tapping task. Journal of NeuroEngineering and Rehabilitation 17:45 (2020), van den Noort et al. Measuring 3D Hand and Finger Kinematics—A Comparison between Inertial Sensing and an Opto-Electronic Marker System. PLoS ONE 11(11): e0164889 (2016), Lee et al. Kinematic Analysis in Patients with Parkinson's Disease and SWEDD. Journal of Parkinson's Disease 4 421-430 (2014), and Heldman et al. The Modified Bradykinesia Rating Scale for Parkinson's Disease: Reliability and Comparison with Kinematic Measures. Mov Disord. 26(10): 1859-1863 (2011) are each incorporated herein by reference.

It should be understood that embodiments in this disclosure are exemplary only, and that other embodiments may include various combinations of features from other embodiments, as well as additional or fewer features.

In general, any process or operation discussed in this disclosure that is understood to be computer-implementable, such as the processes illustrated in the flowcharts disclosed herein, may be performed by one or more processors of a computer system, such as any of the systems or devices in the exemplary environments disclosed herein, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices disclosed herein. One or more processors of a computer system may be included in a single computing device or distributed among a plurality of computing devices. A memory of the computer system may include the respective memory of each computing device of the plurality of computing devices.

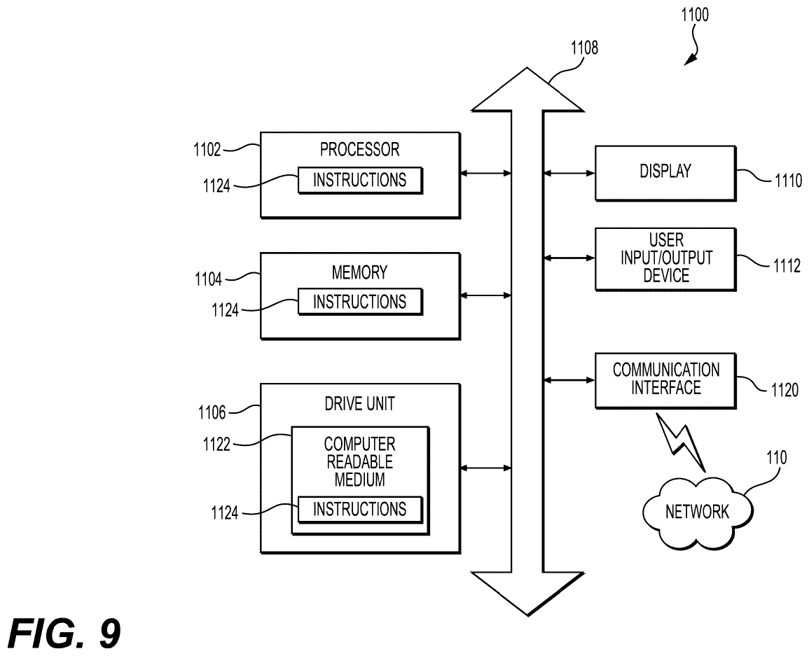

FIG. 9 is a simplified functional block diagram of a computer 1100 that may be configured as a device for executing the methods disclosed here, according to exemplary embodiments of the present disclosure. For example, the computer 1100 may be configured as a system according to exemplary embodiments of this disclosure. In various embodiments, any of the systems herein may be a computer 1100 including, for example, a data communication interface 1120 for packet data communication. The computer 1100 also may include a central processing unit (“CPU”) 1102, in the form of one or more processors, for executing program instructions. The computer 1100 may include an internal communication bus 1108, and a storage unit 1106 (such as ROM, HDD, SDD, etc.) that may store data on a computer readable medium 1122, although the computer 1100 may receive programming and data via network communications (e.g., via network 110). The computer 1100 may also have a memory 1104 (such as RAM) storing instructions 1124 for executing techniques presented herein, although the instructions 1124 may be stored temporarily or permanently within other modules of computer 1100 (e.g., processor 1102 and/or computer readable medium 1122). The computer 1100 also may include input and output ports 1112 and/or a display 1110 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Elements disclosed herein include:
1. A method for motor assessment, the method comprising: receiving first sensed signals in response to a first motor assessment performed using a first test device; receiving second sensed signals in response to a second motor assessment different than the first motor assessment and performed using the first test device; performing noise reduction for the first sensed signals and the second sensed signals; performing position normalization for the first sensed signals and the second sensed signals; generating transformed signals based on the noise reduction and the position normalization for the first sensed signals and the second sensed signals; extracting features based on the transformed signals; providing the extracted features to a machine learning model trained to output a motor assessment based prediction based on the transformed signals; and receiving the motor assessment based prediction from the machine learning model.
2. The method of element 1, wherein one of the first motor assessment or the second motor assessment includes receiving a finger tapping input at the first test device.
3. The method of element 1, wherein one of the first motor assessment or the second motor assessment includes receiving a finger tapping input at the first test device and wherein the finger tapping input is received at a touch screen of the first test device.
4. The method of element 1, wherein one of the first motor assessment or the second motor assessment includes receiving a rotation-based input at the first test device.
5. The method of element 1, wherein one of the first motor assessment or the second motor assessment includes receiving a rotation-based input at the first test device and wherein the rotation-based input includes a pronation component and a supination component.
6. The method of element 1, wherein one of the first sensed signals or the second sensed signals indicate one or more of an area covered, a size, a force, or an impulse.
7. The method of element 1, wherein one of the first sensed signals or the second sensed signals indicate one or more of an angular acceleration, an angular velocity, or a change in magnetic field.
8. The method of element 1, wherein the first sensed signals or the second sensed signals are generated using one or more sensors selected from a force sensor, a touch sensor, an accelerometer, a gyroscope, or a magnetometer.
9. The method of element 1, wherein the first sensed signals or the second sensed signals are generated using an accelerometer, a gyroscope, and a magnetometer.
10. The method of element 1, wherein the first sensed signals or the second sensed signals are generated using an accelerometer, a gyroscope, and a magnetometer and wherein noise reduction is performed based on an accelerometer signal generated at the accelerometer, a gyroscope signal generated at the gyroscope, and a magnetometer signal generated at the magnetometer.
11. The method of element 1, wherein the first motor assessment is performed at a first time and the second motor assessment at a second time different from the first time.
12. The method of element 1, wherein the second motor assessment includes performing multiple motor tasks.
13. The method of element 1, wherein the second motor assessment includes performing multiple motor tasks, the second motor assessment comprising:
performing a first motor task using the first test device at a first time; and
performing a second motor task using a second test device at approximately the first time.
14. The method of element 1, wherein the extracted features identify a patient waveform based on the first motor assessment and the second motor assessment.
15. The method of element 1, wherein the motor assessment based prediction includes a key biomarker, a medical condition, an inclusion criteria, an exclusion criteria, a disease progression attribute, a disease regression attribute, a disease onset, a disease outcome, a disease trend, or a treatment.
16. The method of element 1, wherein one of the noise reduction or the position normalization is performed using a second machine learning model.
17. The method of element 1, wherein the features are extracted using a second machine learning model.
18. A method for motor assessment, the method comprising: receiving first sensed signals in response to a first motor assessment performed using a first test device; receiving second sensed signals in response to a second motor assessment different than the first motor assessment and performed using the first test device; extracting features based on a combination of the first sensed signals and the second sensed signals; providing the extracted features to a machine learning model trained to output a motor assessment based prediction based on the extracted features; and receiving the motor assessment based prediction from the machine learning model.
19. The method of element 18, wherein one of the first motor assessment or the second motor assessment includes receiving a finger tapping input at the first test device.
20. The method of element 18, wherein one of the first motor assessment or the second motor assessment includes receiving a finger tapping input at the first test device and wherein the finger tapping input is received at a touch screen of the first test device.
21. The method of element 18, wherein one of the first motor assessment or the second motor assessment includes receiving a rotation-based input at the first test device
22. The method of element 18, wherein one of the first motor assessment or the second motor assessment includes receiving a rotation-based input at the first test device and wherein the rotation-based input includes a pronation component and a supination component.
23. The method of element 18, wherein one of the first sensed signals or the second sensed signals indicate one or more of an area covered, a size, a force, or an impulse.
24. The method of element 18, wherein one of the first sensed signals or the second sensed signals indicate one or more of an angular acceleration, an angular velocity, or a change in magnetic field.
25. The method of element 18, wherein the first sensed signals or the second sensed signals are generated using one or more sensors selected from a force sensor, a touch sensor, an accelerometer, a gyroscope, or a magnetometer.
26. The method of element 18, wherein the first sensed signals or the second sensed signals are generated using an accelerometer, a gyroscope, and a magnetometer.
27. A system comprising: a data storage device storing processor-readable instructions; and a processor operatively connected to the data storage device and configured to execute the instructions to perform operations that include: receiving first sensed signals in response to a first motor assessment performed using a first test device; receiving second sensed signals in response to a second motor assessment different than the first motor assessment and performed using the first test device; extracting features based on a combination of the first sensed signals and the second sensed signals; providing the extracted features to a machine learning model trained to output a motor assessment based prediction based on the extracted features; and receiving the motor assessment based prediction from the machine learning model.
28. The system of element 27, wherein one of the first motor assessment or the second motor assessment includes receiving a finger tapping input at the first test device.
29. The system of element 27, wherein one of the first motor assessment or the second motor assessment includes receiving a finger tapping input at the first test device and wherein the finger tapping input is received at a touch screen of the first test device.
30. The system of element 27, wherein one of the first motor assessment or the second motor assessment includes receiving a rotation-based input at the first test device.
31. The system of element 27, wherein one of the first motor assessment or the second motor assessment includes receiving a rotation-based input at the first test device and wherein the rotation-based input includes a pronation component and a supination component.
32. The system of element 27, wherein one of the first sensed signals or the second sensed signals indicate one or more of an area covered, a size, a force, or an impulse.
33. The system of element 27, wherein one of the first sensed signals or the second sensed signals indicate one or more of an angular acceleration, an angular velocity, or a change in magnetic field.
34. The system of element 27, wherein the first sensed signals or the second sensed signals are generated using one or more sensors selected from a force sensor, a touch sensor, an accelerometer, a gyroscope, or a magnetometer.
35. The system of element 27, wherein the first sensed signals or the second sensed signals are generated using an accelerometer, a gyroscope, and a magnetometer.
36. A system comprising: a test device comprising a processor; an analysis model; and a machine learning framework trained to output a motor assessment based prediction based on extracted features, wherein the processor is configured to: generate first sensed signals in response to a first motor assessment performed using the test device, and generate second sensed signals in response to a second motor assessment performed using the test device, wherein the analysis model is configured to: extract the extracted features based on a combination of the first sensed signals and the second sensed signals, and provide the extracted features to the machine learning framework, and wherein the machine learning framework is configured to: output the motor assessment based prediction.
37. The system of element 36, wherein one of the first motor assessment or the second motor assessment includes receiving a finger tapping input at the test device.
38. The system of element 36, wherein one of the first motor assessment or the second motor assessment includes receiving a finger tapping input at the test device and wherein the finger tapping input is received at a touch screen of the test device.
39. The system of element 36, wherein one of the first motor assessment or the second motor assessment includes receiving a rotation-based input at the test device.
40. The system of element 36, wherein one of the first motor assessment or the second motor assessment includes receiving a rotation-based input at the test device and wherein the rotation-based input includes a pronation component and a supination component.
41. The system of element 36, wherein one of the first sensed signals or the second sensed signals indicate one or more of an area covered, a size, a force, or an impulse.
42. The system of element 36, wherein one of the first sensed signals or the second sensed signals indicate one or more of an angular acceleration, an angular velocity, or a change in magnetic field.
43. The system of element 36, wherein the first sensed signals or the second sensed signals are generated using one or more sensors selected from a force sensor, a touch sensor, an accelerometer, a gyroscope, or a magnetometer.
44. The system of element 36, wherein the first sensed signals or the second sensed signals are generated using an accelerometer, a gyroscope, and a magnetometer.
45. The system of element 36, wherein the motor assessment based prediction includes a key biomarker, a medical condition, an inclusion criteria, an exclusion criteria, a disease progression attribute, a disease regression attribute, a disease onset, a disease outcome, a disease trend, or a treatment.
46. The system of element 36, wherein the machine learning framework is further configured to output a trigger action, wherein the trigger action includes generating an updated motor assessment, triggering a repeat motor assessment, outputting a treatment, implementing a treatment, or modifying a database.
47. The system of element 46, wherein the machine learning framework includes a first machine learning model configured to extract the extracted features and a second machine learning model configured to output the trigger action.
48. The method of any of elements 1 or 18, wherein the motor assessment based prediction includes a key biomarker, a medical condition, an inclusion criteria, an exclusion criteria, a disease progression attribute, a disease regression attribute, a disease onset, a disease outcome, a disease trend, or a treatment.
49. The system of any of elements 27 or 36, wherein the motor assessment based prediction includes a key biomarker, a medical condition, an inclusion criteria, an exclusion criteria, a disease progression attribute, a disease regression attribute, a disease onset, a disease outcome, a disease trend, or a treatment.
50. The method of any of elements 1 or 18, further comprising outputting a trigger action, wherein the trigger action includes generating an updated motor assessment, triggering a repeat motor assessment, outputting a treatment, implementing a treatment, or modifying a database.

The system of element 27, wherein the operations further include outputting a trigger action, wherein the trigger action includes generating an updated motor assessment, triggering a repeat motor assessment, outputting a treatment, implementing a treatment, or modifying a database.

While the presently disclosed methods, devices, and systems are described with exemplary reference to transmitting data, it should be appreciated that the presently disclosed embodiments may be applicable to any environment, such as a desktop or laptop computer, a mobile device, a wearable device, an application, or the like. Also, the presently disclosed embodiments may be applicable to any type of Internet protocol.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

What is claimed is:

1. A method for motor assessment, the method comprising:
receiving first sensed signals in response to a first motor assessment performed using a first test device;
receiving second sensed signals in response to a second motor assessment different than the first motor assessment and performed using the first test device;
extracting features based on a combination of the first sensed signals and the second sensed signals;
providing the extracted features to a machine learning model trained to output a motor assessment based prediction based on the extracted features; and
receiving the motor assessment based prediction from the machine learning model.

2. The method of claim 1, wherein the extracting features based on the combination of the first sensed signals and the second sensed signals comprises:
performing noise reduction for the first sensed signals and the second sensed signals;
performing position normalization for the first sensed signals and the second sensed signals; and
extracting features based on the combination of the first sensed signals and the second sensed signals based on the noise reduction and the position normalization for the first sensed signals and the second sensed signals.

3. The method of claim 1, wherein the first motor assessment or the second motor assessment includes receiving a finger tapping input at the first test device.

4. The method of claim 1, wherein the first motor assessment or the second motor assessment includes receiving a rotation-based input at the first test device, wherein the rotation-based input includes a pronation component and a supination component.

5. The method of claim 1, wherein the first sensed signals or the second sensed signals indicate one or more of an area covered, a size, a force, or an impulse.

6. The method of claim 1, wherein the first sensed signals or the second sensed signals indicate one or more of an angular acceleration, an angular velocity, or a change in magnetic field.

7. The method of claim 1, wherein the first motor assessment is performed at a first time and the second motor assessment at a second time different from the first time.

8. The method of claim 1, wherein the extracted features identify a patient waveform based on the first motor assessment and the second motor assessment.

9. The method of claim 1, wherein the motor assessment based prediction includes a key biomarker, a medical condition, an inclusion criteria, an exclusion criteria, a disease progression attribute, a disease regression attribute, a disease onset, a disease outcome, a disease trend, or a treatment.

10. The method of claim 1, further comprising outputting a trigger action, wherein the trigger action includes generating an updated motor assessment, triggering a repeat motor assessment, outputting a treatment, implementing a treatment, or modifying a database.

11. A system comprising:
a data storage device storing processor-readable instructions; and
a processor operatively connected to the data storage device and configured to execute the instructions to perform operations that include:
receiving first sensed signals in response to a first motor assessment performed using a first test device;
receiving second sensed signals in response to a second motor assessment different than the first motor assessment and performed using the first test device;
extracting features based on a combination of the first sensed signals and the second sensed signals;
providing the extracted features to a machine learning model trained to output a motor assessment based prediction based on the extracted features; and
receiving the motor assessment based prediction from the machine learning model.

12. The system of claim 11, wherein the first motor assessment or the second motor assessment includes receiving a finger tapping input at the first test device, wherein the finger tapping input is received at a touch screen of the first test device.

13. The system of claim 11, wherein the first motor assessment or the second motor assessment includes receiving a rotation-based input at the first test device and wherein the rotation-based input includes a pronation component and a supination component.

14. The system of claim 11, wherein the first sensed signals or the second sensed signals indicate one or more of an area covered, a size, a force, or an impulse.

15. The system of claim 11, wherein the first sensed signals or the second sensed signals indicate one or more of an angular acceleration, an angular velocity, or a change in magnetic field.

16. The system of claim 11, wherein the first sensed signals or the second sensed signals are generated using one or more sensors selected from a force sensor, a touch sensor, an accelerometer, a gyroscope, or a magnetometer.

17. A system comprising:
a test device comprising a processor;
an analysis model; and
a machine learning framework trained to output a motor assessment based prediction based on extracted features, wherein the processor is configured to:
generate first sensed signals in response to a first motor assessment performed using the test device, and
generate second sensed signals in response to a second motor assessment performed using the test device,
wherein the analysis model is configured to:
extract the extracted features based on a combination of the first sensed signals and the second sensed signals, and
provide the extracted features to the machine learning model, and wherein the machine learning framework is configured to:
output the motor assessment based prediction.

18. The system of claim 17, wherein the motor assessment based prediction includes a key biomarker, a medical condition, an inclusion criteria, an exclusion criteria, a disease progression attribute, a disease regression attribute, a disease onset, a disease outcome, a disease trend, or a treatment.

19. The system of claim 17, wherein the machine learning framework is further configured to output a trigger action, wherein the trigger action includes generating an updated motor assessment, triggering a repeat motor assessment, outputting a treatment, implementing a treatment, or modifying a database.

20. The system of claim 19, wherein the machine learning framework includes a first machine learning model configured to extract the extracted features and a second machine learning model configured to output the trigger action.
